SEQUENCE LISTING (46 sequences)

SEQ ID NO: 1 - DNA, 194 bp, Ixodes ricinus
ataccttccacttgtagcccttcctcatccgatatggtgacggatgccattgcatcctcgtcgtggaagaggtcctcttctaaataagacccatccatatatgtgtgtttgcgaatgccgtcgacgtagctcctgactagaaactcgtcggctaggacagaacttttcttcaggtttagcgtaatgtcctcgtt

SEQ ID NO: 2 - DNA, 607 bp, Ixodes ricinus
  misc_feature 5: /note=“n = A, T, C or G”
taccngggaatccaaaaccaatttttattggaacttccacgtcttcttcaaggcggtggcacctctgcatttatgaagttcgtcttggcattttattttttgcttctttcattgcrgaactcgcaaatgcacttcccgtgcttgtcgcatttcgccccaaaagcgcatggcattccttccggcagattaactttttcaaattcacggttctgaaccaataatagatcgtggcaatgtttgtgctgtttgcgatttgcaaaccagctgtagccaccattggactcaaaggtgcgcacaacatggcgccgaactgtgaaaaacaaattaaggctnctttgtaataacgctagtcttggtacgccgttagaggtcgatgtcgcgcctcgcgattgcaaagtcacttgcacttatcaagctcctggagaaaaatgggtgcaacggggggatcagcgtttgtacttgcaaacatttgtggagacggtaaaccwgtatttcgcggaactcagatgctccagcgtgaagctcgtcttaataaaagttgtaaattcgagtatngatgaagaactgaaattcgaggcatttagaaacaccacgagaagcagcggaa

SEQ ID NO: 3 - DNA, 259 bp, Ixodes ricinus
gatcctacgcctgaaaatgagtgtccatcgtcttcacatagtgccacattgtaattggtacaagctccattttcgtcagcgctgtttgttatgctgccgcctacttttccttcggcactccataagttaaaccctgtcattataagtgtgattgccgtatctcggctgaatgggttccatttttctcttaaataatcacgtgtccatattccatgtattgtgttcatgagtatgtgattctcatcgtatatcttcgcct

SEQ ID NO: 4 - DNA, 170 bp, Ixodes ricinus
ccactcgaaaatggaggctttgaaacatttcagtacccctgtgaactctggctttgcaatgtaacagcaaaaacacttacagttgaagggtgcagtgtcagacgctatggaagttgcatccacgagcacraccctgattactactggccacgttgctrtccgggtcgtcc

SEQ ID NO: 5 - DNA, 168 bp, Ixodes ricinus
gtatgttaccatgtccaacccggttattaaatacaccaagtcgtaggatttgtaggcagctgcattgcccttgacgtactctctcaacgttgccaaggactcaggcccataaatgtagtggggttgaccttgaactcttcgtaaaaagcgttctttctccgtcgtgag

SEQ ID NO: 6 - DNA, 247 bp, Ixodes ricinus
ccgaamataaaacttagtctcaccaatatacgtttgcctaacgcgaaggaacaggcacaaatatactacgagcacgacattctcaagaacacggttcacggagtgtggacgagaattcactcaaaatatccgttccctgaagatgagggaattacactgataatgacagggtttgatttatggagtgccgatttaactgtaggcggcaccataacaaacagcgctgagaaaagcggagcttgtacga

SEQ ID NO: 7 - DNA, 261 bp, Ixodes ricinus
  CDS 1..261: /transl_table=1
atgccttttattttcgtggtgagcttagtcattgtggcctgcatcgtggtagacacagccaaccacaaaggtagagggcggcctgcgaagtgtaaacttcctccggacgacggaccatgcagagcacgaattccgagttactactttgatagaaaaaccaaaacgtgcaaggagtttatgtatggcggatgcgaaggaaacgaaaacaattttgaaaacataactacgtgccaagaggaatgcagagcaaaaaaagtctag

SEQ ID NO: 8 - protein, 86 aa, Ixodes ricinus
  [CDS]1..261 from SEQ ID NO 7
MPFIFVVSLVIVACIVVDTANHKGRGRPAKCKLPPDDGPCRARIPSYYFDRKTKTCKEFMYGGCEGNENNFENITTCQEECRAKKV

SEQ ID NO: 9 - DNA, 292 bp, Ixodes ricinus
catcgmagccatagtatattttgcacttgtcttccgtttcgtcgtagtaggaccgattccacattgtagtacaccagtcacttatatcctgcgggcggtgcttgcatttgtcctgaacaaatcttccacagcgcttgtcgcacgcctcctgggaatagaacgcgttctctcctccgcatctccatttggaatcatagaaacatctttcagtttgaatattgtagcgataataatcggtatcagtttctttgcatggtcctgggaggggtttggcgcaggggccgtattcagg

SEQ ID NO: 10 - DNA, 270 bp, Ixodes ricinus
ggtaatagttgtcaaattccattaatgtatcctgaaatgtgaccatatctttgtttcccctgtaaaatctcataaaaggctgtgtgttttccttaagaagtgtaacagccacgatggtcaatctcacggatggatgtgtgacacttttatatctcaggtttgccgacattgccattacagataaatagttgataatttctttcttgttatagttgtaagcagcgcatgttgttgcatcaagcaccacatgcacttcaggcaatatggttt

SEQ ID NO: 11 - DNA, 316 bp, Ixodes ricinus
agaaagcagtcatattggccatccacaggtcacaatggttctctccttgacctggcatcgggattcgaagtatggtgcagttcacgtagttggaatacaacacgaaatgtgttcgttggtacgccaataggggttctcgcaaagaacatatcatttggaggaaggcgtagtccgtcgagatatcccaaaactagggtttcattgcgtgcgaaccaactgcccccacttctgtatgtgtactgtaaggagtrgttgaacggygtcctctttcccataaccttgaagttttcacactgcagaggattacctctcaaaa

SEQ ID NO: 12 - DNA, 241 bp, Ixodes ricinus
aaggtagcaagggtggtaggctttcctcacaaagagtctggcttccgtgataaccatatccattcctcaccgtatacccgtcatccaacgtcaattgtgttacaaggcagataatgtcaaaatggctctggtccctataatagtcggataatgtagaaatcgctccatgtggccaaatagatgttcctctttcatactgttttaactttaattgtaggtccgcctcgttctcgaggtatgt

SEQ ID NO: 13 - DNA, 636 bp, Ixodes ricinus
  misc_feature 7: /note=“n = A, T, C or G”
ttccccnaattggccttgcganncttgcaagtcgacnctagaggctccgaagatggacagattgcgcatgaaatatttgaaatcgagcagaatggtgattttaggagcgattatattgtgccacccagtttgaaagtgcaagaacgcacagtggtttaccgtaacaagtacaccagagttcctgtaaattttaccgtcgaagttgccatgctgattgataagtatttatacwaggagttcaagaacgagagccacatcgtaccgtacctggctatgatactgactttgataaatctgaggtatgccgacacacatgacccgtacatccagtttcttctcacacaagtgttcgtggggaawwctggcgatcatatgggccacatgcccttccgacgagcgttcttgttcaggcgccggcattatgcgcagtttaggcccaatmacaccttccacttgtaattctccgttgttggatagtgtaagtgaggccattgcatcagcatcgtggaagargccttcctccaagtaggaaccgcccatttaggtttgctttcccaatccgccaatttaanttttaaaaaaaattccccccccaaaaattaattttttttaaaggtggattgtgatttctccgtt

SEQ ID NO: 14 - DNA, 432 bp, Ixodes ricinus
gatcccaaaagtgcccctggarcgacggttacatcatgagctacgtcataaacttcaaaaaccacttcaaattttctccctgctgtgtagaatcaattcgattcgtcgcacgagagcgggactgcctctacaaagtcaatgccaaggatgctgtaaaaagcctaatatctctgcccggatttaggatatcgccaacgagtttctgtcaatttatgcatccgctttaccgcggtgtccatagcgataagaaagcaggtctgtccgattgcgtacagacgtgtagaacggccaaaaatcgacgaggaggctaccattcatggattcacgcggcacttgacggggttccttgcgacaagagaaaccccaagaaggcctgcataaacgggaaatgcaccctccttaagagcatgccccacagaacgtaccgggaat

SEQ ID NO: 15 - DNA, 466 bp, Ixodes ricinus
agggcgttctttgcttyacagggaacrgcatatgggccacgtgaccttccaatgaccgctccaaatctggcataggttgaaytcgcaagtcgtggcgcagcaggcctyccacattcactccatcctcgtcttttaggatgactgccgccatttgttttgtatcgtggtacaggtgtttgttatggtccgagccgtcgacataagtattgaccaacgatcggccgaatgattacggctcaccaaacacatcaaatacccccgtcaagtcaagagctggaagcacaaagcatagtatgtacaagatacccttggaaatctttcccgaagttcaccttgtggtggacagcacatttgccaaagcttttaaatttgacgtgtacaaagtaacgcgttacttcgcagtgcttacaaatgcggctaatcttaggtatgccagcttcgtatttccaaaagtacagctcaggat

SEQ ID NO: 16 - DNA, 377 bp, Ixodes ricinus
ctcgtccacacattctcctaaaatgcaagccttttttttcccacaaggtgtaccgtcgactacactgagtctccaataaatatgttttccggtgcaatttaccttgcagtctttgacgccgtatgtagggtcagcgtgcatgccttcgtcgtacatatacaccctctgacagtagttgctcagtgttgtcatcctaccaggaagcttagacgaacgttttattgtttttgtcgtgtatcgttctctaaggcatttgaattccggacggttgtagaggttcctgacttctcgctggcagcaataagagaactgatactggcgctcgtcttgcatcttgtaactcatgaggtatccgtcatcccatgggcagtccgcag

SEQ ID NO: 17 - DNA, 1670 bp, Ixodes ricinus
  CDS 54..1520: /transl_table=1
aaggaagaagttaggcgtaggctttgggaaaccggtcatcctcgaaaccagagatgtcgggactcagcctgaaattgtggattgtagcgttcttttctttctgcttggccgagaaagagcatgggatcgtgtaccccaggatgcttgaaagcagagcagcaactggagagagaatgcttaaaatcaacgatgacctgacgttgacgctgcagaagagtaaggtcttcgctgacgactttctcttcagcacgaccgacggaattgaacctattgattactacatcaaagccgaagacgctgaacgtgacatctaccacgacgcaactcacatggcatcagtaagggtaacggacgatgatggcgtggaagtggaaggaattcttggagagaggcttcgtgttaaacctttgccggcaatggcccgcagcagcgatggcctcagaccgcatatgttgtacgaagtcgacgcacacgaaaacggccggccacatgattatggttcaccgaacacaacaaatacccccgtagagagaagagctggaggcacagaaccccagatgtacaagataccagcggaaatctatcccgaagtttaccttgtggcggatagtgcctttgccaaagaatttaactttgatgtgaacgccgttacgcgttacttcgcagtgcttacaaatgcggctaatcttaggtatgaaagcttcaaatctccaaaggtacagctcaggatcgttggcataacgatgaacaaaaacccagcagacgagccatacattcacaatatacggggatatgagcagtaccggaatattttgtttaaggaaacactggaggatttcaacactcagatgaagtcaaaacatttttatcgtactgccgatatcgtgtttctcgtgacagcaaaaaatatgtccgaatgggttggtagcacactacaatcatggactggcgggtacgcttacgtaggaacagcgtgttccgaatggaaagtaggaatgtgtgaagaccgaccgacaagctattacggagcttacgttttcgcccatgagctggcgcataatttgggttgtcaacacgatggagatggtgccaatagctgggtgaaagggcacatcggatctgcggactgcccatgggatgacggataccttatgagctacaagatggaagacgagcgccagtataagttttctccctactgccagagagaagtcaggaacctctacaggcgtccggaattcaaatgcctcactgaacgaaaagcgaaaaaaacaatccgctcgtctaagctacctggtgtgatgacatcatcgagcaactattgccggagggtgtacatgtacgaaaaaggcatgcacgccgacgaggcatatggcgtcaaggactgcagggtaaaatgcaccaccacatcaagaatgtattggctactcggtgtagtcgacggtacaccttgcggaaatggaaaggcttgcattcttgggaaatgcaggaacaaaatcaaaataagcaagaaggactgagaggttgataatatcaaattaatcatgatatttcaaccacatgacttcgtgctcaactggtagccccaaataaattttaaaaaaaatcccaatatgcgtggtagaaaaagcagcaaacaataaaacttctaaaaatgtcttgcaaaaatg

SEQ ID NO: 18 - protein, 488 aa, Ixodes ricinus
  [CDS]54..1520 from SEQ ID NO 17
MSGLSLKLWIVAFFSFCLAEKEHGIVYPRMLESRAATGERMLKINDDLTLTLQKSKVFADDFLFSTTDGIEPIDYYIKAEDAERDIYHDATHMASVRVTDDDGVEVEGILGERLRVKPLPAMARSSDGLRPHMLYEVDAHENGRPHDYGSPNTTNTPVERRAGGTEPQMYKIPAEIYPEVYLVADSAFAKEFNFDVNAVTRYFAVLTNAANLRYESFKSPKVQLRIVGITMNKNPADEPYIHNIRGYEQYRNILFKETLEDFNTQMKSKHFYRTADIVFLVTAKNMSEWVGSTLQSWTGGYAYVGTACSEWKVGMCEDRPTSYYGAYVFAHELAHNLGCQHDGDGANSWVKGHIGSADCPWDDGYLMSYKMEDERQYKFSPYCQREVRNLYRRPEFKCLTERKAKKTIRSSKLPGVMTSSSNYCRRVYMYEKGMHADEAYGVKDCRVKCTTTSRMYWLLGVVDGTPCGNGKACILGKCRNKIKISKKD

SEQ ID NO: 19 - DNA, 158 bp, Ixodes ricinus
caccagtgatgcttattgttgcactgcacttgttgataatatccggtcgtcgaattgcacttcggaacttccactccaacttggcgagccgtggattttgacttctcgtgatgctccaccagacagttgcaggacttcagctgcctagatggagcctt

SEQ ID NO: 20 - DNA, 146 bp, Ixodes ricinus
  misc_feature 41: /note=“n = A, T, C or G”
ctgttgttgaactgaaataaataacaaaaaaatcataaagntggaggaaagatgatcgantccccgccccttgacaatcgtccgataaaaaccaactatattcngtcctttttacaaacaattccaantgtctgaccgaaccgcga

SEQ ID NO: 21 - DNA, 140 bp, Ixodes ricinus
  misc_feature 3: /note=“n = A, T, C or G”
ctnggacgangtcctatgacttgcgcttangtttcttagtcttcttcggtttcttctttttttgcttcggtttttcggtgggcgcaggtgtatagtcatcagtgtcggtgggcccatccgaatgagttgtcaaatgacat

SEQ ID NO: 22 - DNA, 143 bp, Ixodes ricinus
tgccgaaaaataacgatgatttgacgttgactctgcagaagagtaaggttttcaccgacagttttctgtttagcacgacgaaggataacgagcctatcgattactacgtgagagccgaagatgccgaacgagacatatatcac

SEQ ID NO: 23 - DNA, 140 bp, Ixodes ricinus
  misc_feature 112: /note=“n = A, T, C or G”
tgttgctacagactcgacgtttcgagcttgctcgccatttmaagacaacgcactcacagaatatttaagtgcgttcgtgawagctgtgggcttacgattgcaggcgcttcantcaccagctgtgatattamagttcctag

SEQ ID NO: 24 - DNA, 144 bp, Ixodes ricinus
tcacgatagttgaaacgttgaaacttgaaatactcccacagtcgttggatgcttcagaactgctaagaacttcacactttgcaagaagtwccaaaatgaaagccgcgatgaccgatgatttagcttccatcttctatcacttga

SEQ ID NO: 25 - DNA, 95 bp, Ixodes ricinus
gaccaccccgtccgaacttgctaaakcaagcaatggagtgaggtgttctatgcgggttgattacaccaatggcgctgcgtggtgcgtggtgattt

SEQ ID NO: 26 - DNA, 1414 bp, Ixodes ricinus
  CDS 143..1276: /transl_table=1
gtagggccgtgcaagcgaaggcagcgaaggctgcgagtgtacgtgcagttcggaagtgcaatatcctgttattaagctctaattagcacactgtgagtcgatcagaggcctctcttaacgccacattgaaaaaggatccaagatggaggcaagtctgagcaaccacatccttaacttctccgtcgacctatacaagcagctgaaaccctccggcaaagacacggcaggaaacgtcttctgctcaccattcagtattgcagctgctctgtccatggccctcgcaggagctagaggcaacactgccaagcaaatcgctgccatcctgcactcaaacgacgacaagatccacgaccacttctccaacttcctttgcaagcttcccagttacgccccagatgtggccctgcacatcgccaatcgcatgtactctgagcagaccttccatccgaaagcggagtacacaaccctgttgcaaaagtcctacgacagcaccatcaaggctgttgactttgcaggaaatgccgacagggtccgtctggaggtcaatgcctgggttgaggaagtcaccaggtcaaagatcagggacctgctcgcacctggaactgttgattcatcgacatcacttatattagtgaatgccatttacttcaaagttgattcatcgacatcacttatattagtgaatgccatttacttcaaatttcacttgacaccacagacctcaaagaaagtggacatgatgcaccaggaaggggacttcaagatgggtcactgcagcgacctcaaggtcactgcgcttgagataccctacaaaggcaacaagacgtcgatggtcattctcctgcccgaagatgtagagggactctcagtcctggaggaacacttgaccgctccgaaactgtcggctctgctcggcggcatgtatgcgacgtccgatgtcaacttgcgcttgccgaagttcaaactagagcagtccataggtttgaaggatgtactgatggcgatgggagtcaaggatttcttcacgtcccttgcagatctttctggcatcagcgctgcggggaatctgtgcgcttcggatgtcatccacaaggcttttgtggaagttaatgaggagggcacagaggctgcagctgccactgccatacccattatgttgatgtgtgcgagatttccacaggtggtgaactttttcgttgaccgcccattcatgttcttgatccacagccatgatccagatgttgttctcttcatgggatccatccgtgagctctaaaaagcatattcttaacggcggccaatcagtctgtggagttatctcttagtcactaatgtgtaacaattctgcaatattcagcttgtgtatttcagtaacttgctagatctttgtgttgttgatgttaggcttcttgcg

SEQ ID NO: 27 - protein, 377 aa, Ixodes ricinus
  [CDS]143..1276 from SEQ ID NO 26
MEASLSNHILNFSVDLYKQLKPSGKDTAGNVFCSPFSIAAALSMALAGARGNTAKQIAAILHSNDDKIHDHFSNFLCKLPSYAPDVALHIANRMYSEQTFHPKAEYTTLLQKSYDSTIKAVDFAGNADRVRLEVNAWVEEVTRSKIRDLLAPGTVDSSTSLILVNAIYFKVDSSTSLILVNAIYFKFHLTPQTSKKVDMMHQEGDFKMGHCSDLKVTALEIPYKGNKTSMVILLPEDVEGLSVLEEHLTAPKLSALLGGMYATSDVNLRLPKFKLEQSIGLKDVLMAMGVKDFFTSLADLSGISAAGNLCASDVIHKAFVEVNEEGTEAAAATAIPIMLMCARFPQVVNFFVDRPFMFLIHSHDPDVVLFMGSIREL

SEQ ID NO: 28 - DNA, 200 bp, Ixodes ricinus
accgtaaccaaaattgtttctttccagaagaatggttcaaacttttcaaacagatttcggaaactcttcttgcacttttaaaatccaatctacaatctttcctcgcacttctgaattgcattccagtttaccttccaagcaaacctcttttggcaactccagccgtactccatttcggcataccacagtgcatgcacttg

SEQ ID NO: 29 - DNA, 241 bp, Ixodes ricinus
cgtattctttgaagatttgtatacgaaacataaattcgtcatgcatacttttgatggttacacgacatgcgaagctgccgacaaagaagactgggaagataagaagcacctagttacggtagtgcgtggaccggataaacgaaagtacacgtttctacgcaacattctcaccttacaacggagagtgagagttagcaaaacaatgattgagctcgtacggaacatgtcctgtaggacattt

SEQ ID NO: 30 - DNA, 313 bp, Ixodes ricinus
  misc_feature 6: /note=“n = A, T, C or G”
aagcanccggactacctgcttgaaaacgttgtacgggcaaacttggacggaaaactcccagatgctactccagttcctcccggaagctacacgtacgctgagaatgataacttcacctgctattccagaagtacaccgtttccggatggggtgaatgttgtataacggctgctgggtgcggaagactatgatggattacgcaaaaaagttctaaacgagttgtttcccatcccggaaagtctgctgtatgctgacatgatgcgacttgtggctaagaaagacagagttgatcacactagtggatgacctggga

SEQ ID NO: 31 - DNA, 2417 bp, Ixodes ricinus
  CDS 218..1495: /transl_table=1
gtcgtagtcgtagtcgtagtcagttgcgcatgcgcggggctttcctgtctttcttgcctttctgcagtcgttcaccaacatgtggatacagctccggagatttgtaaacaaatactgcacttttaagcaagacttgatatttagatcgatatcctcctgttgtccgtcttgattaatcggctctttagggtttttagaataggcttttcggtacgagatgcccaaaggaaagaggggacccaaagcaggtggcgccgcgcgcggtggccggtgcgaggccagcctggctccgtcgtccagcgacgaggagtccaacgcagacacggcgagcgtgctgagctgcgcctcggagtctcgctgtggcagtgacggcaccgttggagacccagaagcggaggaggctgtgctgcatgacgactttgaagacaaactcaaggaggccatcgacggagcttcgcagaagagtgccaaaggacggctgtcgtgcctggaggcgattcgcaaggccttttccaccaaatacctgtacgacttcctcatggacagaccgagcacggtgtgcgacctggtggagcgtggggtgcgcaagggccgaggggaggaggcggccctgtgcgccactctcggggccctggcctgcgtccagctcggggtcggggccgaggcggacgccctgttcgacgccctgcgccagccgctctgcactttgctgcttgacggggcccaggggccctcccccagggccaggtgtgccactgccctcggcctctgctgcttcgtggtggactcggacaaccagctggtgctgcagccgtgcatggaggtgctctggcaggtggtgggtgccaaggcgggccccggctctccggtgctccaggcagcggccctgctcgcctggggcctcctgctcagcgtggctcccgtcgaccgcctgctggcgctcacgcgcacgcacctgccccggctgcaggagctgctggagagccccgacctggacctgcgcattgcggccggggaggtgatcgccgtcatgtacgagggggccagggactacgacgaggactttgaggagccctcggagtccctgtgtgcccagctgcgccagctggccacggacagccagaagtttcgggccaagaaggagcggcgccagcagcgctccaccttcagggacgtctaccgggccgtcagggagggggcctctcccgacgtgagcgtcaagtttggccgggaagtcctggaactggacacctggagtcgcaagctgcagtacgacgctttctgccagctgctgggctccggcatgaacctgcacctggccgtgaacgagctgctgagggacatctttgaactggggcaggtgctggcaaccgaggaccacattatctccaagatcaccaagttcgaaaggcacatggtgaacatggccagctgccgggcccgcaccaagacacgcaaccggctgagggacaagcgcgccgacgtggtcgcctgaacctgcggagggatgcttagctatgcactcgccggcctaccctggcgggactcgatgccactcacgagtcggcgctcgcaaattcgccgcccatcgttacgcaatgggagacaaagctgcttttggcattaccgtttgaggtcggctccaacccatagatgaatttcttttttgtggccgtttctgggttacatgttttgggggaagggagtggaactgtccggttctttggcacacgtcaggttgctcttgatgcgcgacgtgcttgtatttgggtactgccgacaccaagcgtttcggcgattcctggaaaagagtgcctctcgctcgacgtttggttgttttctgcgtggtccgtcgtcgaccttcgttcgtccaaagacgccgtccggtttcatactcccccccgcacacatatcgaggccaattaaattgctaagggtgccgttgtcgtgcatctggcaggctcagaagtggcttatttgtcttttaattttgccgatgcacgcaaaaattgtcatttcttgaaagtttctcttttattgcgtacacaattcaacttttatgtaatttctgatggtctgttttacgtgtgcgtgtgtaaaacgtaactttggaagaatttttatgcacactgaacaaacgctcggtcctggggttgaaagtgctcggtgtgtgcatgagctaaagtgcaactgctttgttccgaaggttttctagtcgccgaaatgtaccattgtggaccttgttgcgagagaccttggtcttctgggggagctgctgtagcgtggcaagccactattttgggagcgacattgcagagaaaatcggcttttagaaaggcacctgcgcggcgagtggacgttttttcgtatatactgcgaaaaaaaaaaaaaaaaaaaaaaaaaaaaaaaaaaaaaaa

SEQ ID NO: 32 - protein, 425 aa, Ixodes ricinus
  [CDS]218..1495 from SEQ ID NO 31
MPKGKRGPKAGGAARGGRCEASLAPSSSDEESNADTASVLSCASESRCGSDGTVGDPEAEEAVLHDDFEDKLKEAIDGASQKSAKGRLSCLEAIRKAFSTKYLYDFLMDRPSTVCDLVERGVRKGRGEEAALCATLGALACVQLGVGAEADALFDALRQPLCTLLLDGAQGPSPRARCATALGLCCFVVDSDNQLVLQPCMEVLWQVVGAKAGPGSPVLQAAALLAWGLLLSVAPVDRLLALTRTHLPRLQELLESPDLDLRIAAGEVIAVMYEGARDYDEDFEEPSESLCAQLRQLATDSQKFRAKKERRQQRSTFRDVYRAVREGASPDVSVKFGREVLELDTWSRKLQYDAFCQLLGSGMNLHLAVNELLRDIFELGQVLATEDHIISKITKFERHMVNMASCRARTKTRNRLRDKRADVVA

SEQ ID NO: 33 - DNA, 933 bp, Ixodes ricinus
  CDS 32..853: /transl_table=1
gattgggaacctcctattcctcacttgaaacatggctggactccgctcctgcatcctcctggctcttgccactagtgccttcgccggctaccttcacggtggccttacccacggcgctgggtacggttacggtgtcggctacggttccggccttggctatggccttggctacggttccggccttggctatggacatgctgttggccttggacacggctttggctattctggtctgaccggctacagtgtggctgccccagctagctacgccgttgctgctccagccgtcagccgcaccgtttccacttaccacgctgctccagctgtggccacctacgccgctgctcctgtcgccacctatgctgttgctccagctgtcactagggtttcccccgttcgcgccgccccagctgtggccacgtacgccgccgctccagtcgccacctacgccgctgctccagctgtgaccagggtgtccaccattcacgctgccccggctgtggccaattacgccgtcgctccagtcgccacctatgccgctgctccagctgtgaccagggtgtccaccatccacgccgctccagccgtggctagctaccagacctaccacgctccagctgtcgccactgtggctcatgctccagctgtggccagctaccagacctaccacgctgccccagccgtggctacctacgcccatgccgctcccgtctacggctatggtgtcggtaccctcggatatggtgtcggccactacggctacggacacggtcttggcagctacggcctgaactacggttacggcctcggcacctacggtgactacaccacccttctccgcaagaagaagtaaatggcacatctcaagagagcccattggactgccatcgacattcttcttcaataaaagagcccgaagatggcattattttt

SEQ ID NO: 34 - protein, 273 aa, Ixodes ricinus
  [CDS]32..853 from SEQ ID NO 33
MAGLRSCILLALATSAFAGYLHGGLTHGAGYGYGVGYGSGLGYGLGYGSGLGYGHAVGLGHGFGYSGLTGYSVAAPASYAVAAPAVSRTVSTYHAAPAVATYAAAPVATYAVAPAVTRVSPVRAAPAVATYAAAPVATYAAAPAVTRVSTIHAAPAVANYAVAPVATYAAAPAVTRVSTIHAAPAVASYQTYHAPAVATVAHAPAVASYQTYHAAPAVATYAHAAPVYGYGVGTLGYGVGHYGYGHGLGSYGLNYGYGLGTYGDYTTLLRKKK

SEQ ID NO: 35 - protein, 90 aa, Ixodes ricinus
MKLTMQLIFVVSLVIVACIVVDTANHKGRGRPAKCKLPPDDGPCRARIPSYYFDRKTKTCKEFMYGGCEGNENNFENITTCQEECRAKKV

SEQ ID NO: 36 - protein, 67 aa, Ixodes ricinus
ANHKGRGRPAKCKLPPDDGPCRARIPSYYFDRKTKTCKEFMYGGCEGNENNFENITTCQEECRAKKV

SEQ ID NO: 37 - protein, 65 aa, Bungarus fasciatus
KNRPTFCNLLPETGRCNALIPAFYYNSHLHKCQKFNYGGCGGNANNFKTIDECQRTCAAKYGRSS

SEQ ID NO: 38 - DNA, 33 bp, Artificial Sequence
  synthetic primer
cgcggatccgcggccaaccacaaaggtagaggg

SEQ ID NO: 39 - DNA, 37 bp, Artificial Sequence
  synthetic primer
ccgctcgagcggttagactttttttgctctgcattcc

SEQ ID NO: 40 - RNA, 19 nt, Artificial Sequence
  synthetic siRNA
ccaugcagagcacgaauuc

SEQ ID NO: 41 - RNA, 19 nt, Artificial Sequence
  synthetic siRNA
gcacgaauuccgaguuacu

SEQ ID NO: 42 - RNA, 19 nt, Artificial Sequence
  synthetic siRNA
acuacgugccaagaggaau

SEQ ID NO: 43 - DNA, 24 bp, Artificial Sequence
  synthetic primer
atgaaactaacgatgcagctgatc

SEQ ID NO: 44 - DNA, 25 bp, Artificial Sequence
  synthetic primer
ttagactttttttgctctgcattcc

SEQ ID NO: 45 - DNA, 21 bp, Artificial Sequence
  synthetic primer
atgtgtgacgacgaggttgcc

SEQ ID NO: 46 - DNA, 22 bp, Artificial Sequence
  synthetic primer
ttagaagcacttgcggtggatg

(19) United States
(12) Patent Application Publication
(10) Pub. No.: US 2016/0046728 A1
(43) Pub. Date: Feb. 18, 2016

(54) IDENTIFICATION AND MOLECULAR CHARACTERISATION OF PROTEINS, EXPRESSED IN THE IXODES RICINUS SALIVARY GLANDS

(71) Applicant: BIOXODES SA, Marche-en-Famenne (BE)

(72) Inventors: Edmond GODFROID, Bruxelles (BE); Yves DECREM, Perwez (BE); Luc VANHAMME, Court-Saint-Etienne (BE); Alex BOLLEN, Ixelles (BE); Gerard LEBOULLE, Berlin (DE); Michel GUYAUX, Bruxelles (BE)

(21) Appl. No.: 14/883,217

(22) Filed: Oct. 14, 2015

Related U.S. Application Data
(63) Continuation-in-part of application No. 14/302,862, filed on Jun. 12, 2014, now Pat. No. 9,212,216.
Application No. 14/302,862 is a division of application No. 13/632,763, filed on Oct. 1, 2012, now abandoned.
Application No. 13/632,763 is a continuation of application No. 11/932,985, filed on Oct. 31, 2007, now Pat. No. 8,277,813.

(51) Int. Cl. C07K 16/40; C07K 14/81; C07K 16/46

(52) U.S. Cl. CPC C07K 16/40; C07K 16/468; C07K 14/8114; C07K 2317/76; C07K 2319/00; C07K 2317/31; C07K 2317/626; C07K 2317/92

(57) ABSTRACT

The invention relates to a new polynucleotide which encodes a polypeptide expressed in the salivary glands of ticks, more particularly the Ixodes ricinus arthropod tick, during the slow-feeding phase of the blood meal have, said polynucleotide and related polypeptide may be used in different constructions and for different applications which are also included in the present invention.

This application is a Continuation-in-Part of U.S. Ser. No. 14/302,862, filed 12 Jun. 2014, which is a divisional of U.S. Ser. No. 13/632,763, filed 1 Oct. 2012, which is a Continuation of U.S. Ser. No. 11/932,985, filed 31 Oct. 2007.

### FIELD OF THE INVENTION

The present invention is related to the molecular characterisation of DNA sequences, which encode proteins expressed in the salivary glands of the Ixodes ricinus arthropod tick. These proteins are involved in the complex mechanism of interaction between this arthropod and its mammalian host. The invention relates to newly identified polynucleotides, polypeptides encoded by them and the use of such polynucleotides and polypeptides, and to their production.

### BACKGROUND OF THE INVENTION

Ticks are hematophagous arthropods that feed on a wide diversity of hosts. Unlike this group of arthropods, the Ixodid adult female ticks have the characteristics to ingest blood for an extended period of over 2 weeks.

Completion of the blood meal is dependent on the relationships of ticks with hosts species. Resistance to tick infestation implicates both innate and acquired immunity, and is characterized by reduced feeding, molting and mating capabilities that may lead to the death of the parasite. Acquired immunity of resistant hosts is mediated by a polarized Th1-type immune response, involving IFN-α production and delayed type hypersensitivity reaction.

Some hosts are unable to counteract the tick infestation. Indeed, during their blood meal, ticks circumvent host defences via pharmacologically active components secreted in their saliva. These factors can modulate both the innate and the acquired immunity of the host. In this way, the leukocyte responsiveness is modified during tick feeding. For example, cytokines production is modulated, inducing a polarised Th2 immune response.

Therefore, the complex tick-host molecular interaction can be considered as a balance between host defences raised against the parasite and the tick evasion strategies, facilitating feeding for an extended period. Although, there is extensive information about the effects of tick bioactive factors on host immune defences, little is known about the mechanisms of their actions. However, it has been observed that a wide range of new proteins is expressed during the blood meal. Several of them might be essential for the completion of the tick feeding process.

### SUMMARY OF THE INVENTION

The present invention relates to an inhibitor of a plasma contact factor, wherein said inhibitor is an isolated polypeptide having less than 100% and at least 75% sequence identity to the amino acid SEQ ID NO: 36 or a diabody.

In one embodiment, said inhibition is selected from the group comprising inhibition of the activation of factor XI into factor XIa by factor XIIa, inhibition of the activation of factor XII into factor XIIa by factor XIa, or a combination thereof.

In one embodiment, said isolated polypeptide comprises at least 80% sequence identity to the amino acid SEQ ID NO: 36. In another embodiment, said isolated polypeptide comprises at least 90% sequence identity to the amino acid SEQ ID NO: 36. In another embodiment, said isolated polypeptide comprises at least 95% sequence identity to the amino acid SEQ ID NO: 36.

In one embodiment, said isolated polypeptide comprises at least one substitution group. In a particular embodiment, said isolated polypeptide is selected from the group consisting of a polypeptide having up to 5 amino acids substitutions relative to the amino acid sequence of SEQ ID NO: 36, a polypeptide having up to 5 amino acids deletions relative to the amino acid sequence of SEQ ID NO: 36, and a polypeptide having up to 5 amino acids additions relative to the amino acid sequence of SEQ ID NO: 36.

In one embodiment, said isolated polypeptide is a polypeptide having at least 95% sequence identity to the amino acid sequence of SEQ ID NO:36, wherein said polypeptide has a kunitz-type-protease-inhibitor (KPI) domain, wherein the KPI domain of the polypeptide comprises Phe at position corresponding to position 40 of SEQ ID NO:36, Gly at position corresponding to position 44 of SEQ ID NO:36; Cys at position corresponding to position 45 of SEQ ID NO:36, Phe at position corresponding to position 52 of SEQ ID NO:36, and Cys at position corresponding to position 58 of SEQ ID NO:36.

According to one embodiment, said isolated polypeptide is fused to a heterologous polypeptide. In one embodiment, said heterologous polypeptide comprises multiple histidine residues.

In one embodiment, said isolated polypeptide is a polypeptide having at least 95% sequence identity to the amino acid sequence of SEQ ID NO:36 fused to a heterologous polypeptide, wherein said polypeptide has a kunitz-type-protease-inhibitor (KPI) domain, wherein the KPI domain of the polypeptide comprises Phe at position corresponding to position 40 of SEQ ID NO:36, Gly at position corresponding to position 44 of SEQ ID NO:36; Cys at position corresponding to position 45 of SEQ ID NO:36, Phe at position corresponding to position 52 of SEQ ID NO:36, and Cys at position corresponding to position 58 of SEQ ID NO:36.

In one embodiment, the diabody of the invention recognizes two different polypeptides from the group comprising factor XI, factor XII, factor XIa and factor XIIa.

Another object of the present invention is a method for preventing and/or treating a plasma contact factor-related disease comprising administration of an inhibitor of a plasma contact factor in a subject in need thereof, wherein said inhibitor is an isolated polypeptide having less than 100% and at least 75% sequence identity to the amino acid SEQ ID NO: 36 or a diabody.

In one embodiment, said plasma contact factor-related disease is selected from the group comprising deep vein thrombosis, portal vein thrombosis, jugular vein thrombosis, renal vein thrombosis, pulmonary embolism, unstable angina, acute coronary syndrome, myocardial infraction, cerebral ischemia and stroke.

In another embodiment, said plasma contact factor-related disease is the thrombus formation during and/or after the contact of blood with artificial surfaces.

In another embodiment, said plasma contact factor-related disease is the thrombus formation during and/or after a medical procedure such as comprising extracorporeal membrane oxygenation for blood oxygenation, extracorporeal circulation during cardiopulmonary bypass, dialysis and extracorporeal filtration of blood, percutaneous angioplasty, use intraluminal catheters and stents, intra-aortic balloon pump.

### BRIEF DESCRIPTION OF THE DRAWINGS

FIG. 1 shows sequencing of SEQ ID NO: 24 and 16.

FIG. 2 shows that the expression of the selected sequences is induced in salivary glads of 5 day engorged ticks, except for the sequence 31 that is expressed at a similar level in salivary glads of engorged and unfed ticks.

FIG. 3A represents amino acid sequence of Ir-CPI with its peptide signal (SEQ ID NO: 35). FIG. 3B represents comparison of SEQ ID NO: 36 (Ir-CPI) with the kunitz-type chymotrypsin inhibitor from Bungarus fasciatus (BF9) (SEQ ID NO: 37). Some shared conserved residues are shaded (P, proline residue; G, glycine residue). Three disulphide bridges are represented. Finally, no consensus sites for N- and 0-glycosylation were predicted in the sequence.

FIGS. 4A-4C represent the effects of Ir-CPI on aPTT, PT and Fibrinolysis times. Inhibitory activities of Ir-CPI was estimated on the intrinsic and extrinsic coagulation pathways, and on fibrinolysis.

FIG. 5A represents evaluation of the Ir-CPI siRNA specificity by RT-PCR. FIG. 5B represents the effect of Ir-CPI siRNA-treated salivary glad extracts on aPTT and PT.

FIGS. 6A-6B represent the effect of Ir-CPI on thrombin activity profile during coagulation process induced by either ellagic acid and PL (A) or 5 pM TF and PL (B).

FIG. 7 represents the inhibitory effect of Ir-CPI on generation of factor XIIa, factor Xia and kallikrein in human plasma.

FIGS. 8A-8D represent the inhibitory effect of Ir-CPI on reconstituted systems.

FIG. 9 represents sensorgrams for interactions between coagulation factors and immobilized Ir-CPI measured by surface plasmon resonance.

FIG. 10 represents inhibitory effect of Ir-CPI on the activation of the classical complement pathway by fragment f of factor XII (factor Hf).

FIG. 11 represents the effect of Ir-CPI on stasis-induced venous thrombosis in rats.

FIGS. 12A-12C represent ex vivo aPTT (A), PT (B) and fibrinolysis (C) activities of Ir-CPI.

FIG. 13 represents the determination of the bleeding effect of Ir-CPI. Ir-CPI at the indicated dose was administered intravenously; after 5 min of administration, the rat tail was cut 3 mm from the tip.

FIGS. 14A-14B represent the inhibition of factor XI (A) and XII (B) coagulation activities. Experiments were repeated three times independently.

FIGS. 15A-15C represent the activation and amplification of the intrinsic coagulation pathway (A), targets of Ir-CPI (B) and targets of bispecific diabody (C).

### DEFINITIONS

“Putative anticoagulant, anti-complementary and immunomodulatory” polypeptides refer to polypeptides having the amino acid sequence encoded by the genes indicated in the table. These present homologies with anticoagulant, anti-complementary and immunomodulatory polypeptides already existing in databases. These polypeptides belong to the Class I and Class II sequences (see table).
“Putative anticoagulant, anti-complementary and immunomodulatory” cDNAs refer to polynucleotides having the nucleotide sequence described in the table, or allele variants thereof and/or their complements. These present homologies with anticoagulant, anti-complementary and immunomodulatory polynucleotides already existing in databases. These cDNAs belong to the Class I and Class II sequences (see table).
Some polypeptide or polynucleotide sequences present low or no homologies with already existing polypeptides or polynucleotides in databases. These belong to the Class III (see table).
“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications include acetylation, acylation, ADP-ribosylation, amidation, covalent attachment of flavin, covalent attachment of a hem moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-linkings, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino of amino acids to proteins such as arginylation, and ubiquitination. See, for instance, PROTEINS—STRUCTURE AND MOLECULAR PROPERTIES, 2nd Ed., T. E. Creighton, W. H. Freeman and Comany, New York, 1993 and Wolt, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182: 626-646 and Rattan et al, “Protein Synthesis: Posttranslational Modifications and Aging”, Ann NY Acad Sci (1992) 663: 48-62.
“Polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Polynucleotide” refers to triple-stranded regions comprising RNA or DNA or both RNA and DNA. The term “Polynucleotide” also includes dNas or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “Polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.
“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions (preferably conservative), additions and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide. For instance, they should have similar antigenic or immunogenic activities as the reference polypeptide. Antigenicity can be tested using standard immunoblot experiments, preferably using polyclonal sera against the reference polypeptide. The immunogenicity can be tested by measuring antibody responses (using polyclonal sera generated against the variant polypeptide) against purified reference polypeptide in a standard ELISA test. Preferably, a variant would retain all of the above biological activities.
“Identity” is a measure of the identity of nucleotide sequences or amino acid sequences. In general, the sequences are aligned so that the highest order match is obtained. “Identify” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds, Humana Press, New Jersey, 1994; SEQUENCE ANALYSIS IN MOLECULAR BIOLOGY, von Heijne, G., Academic Press, 1987; and SEQUENCE ANALYSIS PRIMER, Gribskov, M. and Devereux, J., eds, M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1998) 48: 1073). Methods commonly employed to determine identity or similarity between two sequences include, but are not limited to those disclosed in Guide to Huge Computers, Martin J. Bishop, ed., Academic Press, San Diego, 1994, and Carillo, H., and Lipton, D., SIAM J Applied Math (1988) 48: 1073. Methods to determine identity and similarity are codified in computer programs. Preferred computer program methods to determine identity and similarity between two sequences include, but are not limited to, GCG program package (Devereux, J., et al., J Molec Biol (1990) 215: 403). Most preferably, the program used to determine identity levels was the GAP program, as was used in the Examples hereafter.
As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.
Fragments of I. ricinus salivary gland polypeptides are also included in the present invention. A fragment is a polypeptide having an amino acid sequence that is the same as a part, but not all, of the amino acid sequence of the aforementioned I. ricinus salivary gland polypeptides. As with I. ricinus salivary gland polypeptides, fragment may be “free-standing” or comprised within a larger polypeptide of which they form a part or region, most preferably as a single continuous region. Representative examples of polypeptide fragments of the invention, include, for example, fragments from about amino acid number 1-20, 21-40, 41-60, 61-80, 81-100, and 101 to the end of the polypeptide. In this context “about” includes the particularly recited ranges larger or smaller by several, 5, 4, 3, 2 or 1 amino acid at either extreme or at both extremes.
Preferred fragments include, for example, truncated polypeptides having the amino acid sequence of the I. ricinus salivary gland polypeptides, except for deletion of a continuous series of residues that includes the amino terminus, or a continuous series of residues that includes the carboxyl terminus and/or transmembrane region or deletion of two continuous series of residues, one including the amino terminus and one including the carboxyl terminus. Also preferred are fragments characterised by structural or functional attributes such as fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate I. ricinus salivary gland protein activity, including those with a similar activity or an improved activity, or with a decreased undesirable activity. Also included are those that are antigenic or immunogenic in an animal or in a human.
“Diabody” refers to small antibody fragments prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993). As used herein, a diabody according to the invention may be also called a bispecific antibody.
“Preventing a disease” refers to keeping from happening at least one adverse effect or symptom of a disease, disorder or condition associated with a deficiency in or absence of an organ, tissue or cell function. According to the invention, the term “treating a disease” refers to reducing or alleviating at least one adverse effect or symptom of a disease, disorder or condition associated with a deficiency in an organ, tissue or cell function.
“Pharmaceutical composition” refers to a composition comprising an active principle in association with a pharmaceutically acceptable vehicle or excipient. A pharmaceutical composition is for therapeutic use, and relates to health. Especially, a pharmaceutical composition may be indicated for treating or preventing a disease. According to the invention, the term “treating a disease” refers to reducing or alleviating at least one adverse effect or symptom of a disease, disorder or condition associated with a deficiency in an organ, tissue or cell function. The expression “Preventing a disease” or “Inhibiting the development of a disease” refers to preventing or avoiding the occurrence of symptom.
“Excipient” refers to a substance that, by its addition into a composition comprising a compound of interest, allows to obtain a desired consistency or other physical characteristics, whilst avoiding any interaction with the compound of interest, in particular chemical interaction.
“Pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA.
“Subject” refers to a mammal, preferably a human. In one embodiment, a subject may be a “patient”, i.e. a warm-blooded animal, more preferably a human, who/which is awaiting the receipt of, or is receiving medical care or was/is/will be the object of a medical procedure, or is monitored for the development of a disease.

### DETAILED DESCRIPTION

The present invention relates to an inhibitor of a plasma contact factor.

In one embodiment, the inhibitor of a plasma contact factor of the invention inhibits the the activation of factor XI into factor XIa by factor XIIa, or the activation of factor XII into factor XIIa by factor XIa, or a combination thereof. In a preferred embodiment, the inhibitor of a plasma contact factor of the invention inhibits the activation of factor XI into factor XIa by factor XIIa and the activation of factor XII into factor XIIa by factor XIa.

In other words, in one embodiment of the invention, the inhibition by the inhibitor of the invention is selected from the group comprising the inhibition of the activation of factor XI into factor XIa by factor XIIa and the inhibition of the activation of factor XII into factor XIIa by factor XIa, or a combination thereof. In a preferred embodiment, the inhibition of the invention is a combination of the inhibition of the activation of factor XI into factor XIa by factor XIIa and the inhibition of the activation of factor XII into factor XIIa by factor XIa.

In one embodiment, the inhibitor of the invention is an isolated polypeptide comprising an isolated polypeptide having the amino acid sequence SEQ ID NO: 36. In another embodiment, the inhibitor of the invention is an isolated polypeptide consisting on the isolated polypeptide having the amino acid sequence SEQ ID NO: 36.

In one embodiment, the inhibitor of the invention is an isolated polypeptide having less than 100% and at least 75% sequence identity to the amino acid sequence SEQ ID NO: 36. In one embodiment, the inhibitor of the invention is an isolated polypeptide having at least 80% sequence identity to the amino acid sequence SEQ ID NO: 36, preferably at least 90% sequence identity, more preferably at least 95% sequence identity.

In one embodiment, the isolated polypeptide of the invention comprises at least one substitution group.

In another embodiment, the isolated polypeptide of the invention is selected from the group consisting of a polypeptide having up to 3 amino acids substitutions relative to the amino acid sequence of SEQ ID NO: 36, a polypeptide having up to 3 amino acids deletions relative to the amino acid sequence of SEQ ID NO: 36, and a polypeptide having up to 3 amino acids additions relative to the amino acid sequence of SEQ ID NO: 36.

In one embodiment, the isolated polypeptide of the invention is selected from the group consisting of a polypeptide having up to 5 amino acids substitutions relative to the amino acid sequence of SEQ ID NO: 36, a polypeptide having up to 5 amino acids deletions relative to the amino acid sequence of SEQ ID NO: 36, and a polypeptide having up to 5 amino acids additions relative to the amino acid sequence of SEQ ID NO: 36.

In one embodiment, the isolated polypeptide of the invention has a kunitz-type-protease-inhibitor (KPI) domain. In one particular embodiment, the KPI domain of the polypeptide comprises Phe at position corresponding to position 40 of SEQ ID NO: 36, Gly at position corresponding to position 44 of SEQ ID NO: 36; Cys at position corresponding to position 45 of SEQ ID NO: 36, Phe at position corresponding to position 52 of SEQ ID NO: 36, and Cys at position corresponding to position 58 of SEQ ID NO: 36.

In one embodiment, the isolated polypeptide of the invention is a polypeptide having at least 95% sequence identity to the amino acid sequence of SEQ ID NO:36, wherein said polypeptide has a kunitz-type-protease-inhibitor (KPI) domain, wherein the KPI domain of the polypeptide comprises Phe at position corresponding to position 40 of SEQ ID NO:36, Gly at position corresponding to position 44 of SEQ ID NO:36; Cys at position corresponding to position 45 of SEQ ID NO:36, Phe at position corresponding to position 52 of SEQ ID NO:36, and Cys at position corresponding to position 58 of SEQ ID NO:36.

In one embodiment, the isolated polypeptide of the invention isolated polypeptide is fused to a heterologous polypeptide. In a particular embodiment, the isolated polypeptide of the invention heterologous polypeptide comprises multiple histidine residues.

In one embodiment, the isolated polypeptide of the invention is a polypeptide having at least 95% sequence identity to the amino acid sequence of SEQ ID NO:36 fused to a heterologous polypeptide, wherein said polypeptide has a kunitz-type-protease-inhibitor (KPI) domain, wherein the KPI domain of the polypeptide comprises Phe at position corresponding to position 40 of SEQ ID NO:36, Gly at position corresponding to position 44 of SEQ ID NO:36; Cys at position corresponding to position 45 of SEQ ID NO:36, Phe at position corresponding to position 52 of SEQ ID NO:36, and Cys at position corresponding to position 58 of SEQ ID NO:36.

Other compounds having the property of inhibiting both factor XI and XII may be useful for inhibiting thrombus (clot) and/or coagulation. Dual or bispecific recognition of two molecular targets can rationally be obtained using the diabody technology (Holliger et al., ““Diabodies”: small bivalent and bispecific antibody fragments”, Proc. Natl. Acad. Sci. USA. (1993) 90: 6444-6448; Spiess et al. “Alternative molecular formats and therapeutic applications for bispecific antibodies”, Molecular Immunology (2015) 67: 95-106). This technology has been used for creating bispecific functional antibodies against cytokine(s), receptor(s), growth factors and co-stimulatory/inhibitory surface receptors with potential therapeutic applications mainly in the field of oncology and immunology.

Therefore, in another embodiment, the inhibitor of the invention is a diabody.

In one embodiment, the diabody of the invention is a heterodimer diabody, i.e. a bispecific antibody. In one embodiment, the heterodimer diabody, or bispecific antibody, of the invention has two different antigen-binding sites, wherein each of them recognizes one polypeptide from the group comprising factor XI, factor XII, factor XIa and factor XIIa. In other words, according to one embodiment, factor XI, factor XII, factor XIa and/or factor XIIa are ligands of the diabody of the invention.

Examples of diabodies of the invention include, but are not limited to, diabodies having an antigen-binding site recognizing factor XI and an antigen-binding site recognizing factor XII, diabodies having an antigen-binding site recognizing factor XIa and an antigen-binding site recognizing factor XIIa, diabodies having an antigen-binding site recognizing factor XIa and an antigen-binding site recognizing factor XII or diabodies having an antigen-binding site recognizing factor XI and an antigen-binding site recognizing factor XIIa.

Diabodies may be classified in two categories, agonist diabodies and antagonist diabodies. In one embodiment, the diabody of the invention is an antagonist diabody, i.e. a diabody which inhibits its ligands.

Diabodies of the invention may be prepared according to any method known in the art. Examples of methods for the preparation of diabodies include, but are not limited to, preparation from bacterial periplasmic fraction using a co-expression vector (i.e. genes encoding two chains were tandemly located under the same promoter).

In one embodiment, the inhibitor of a plasma contact factor of the invention binds to factor XI and/or factor XII. In another particular embodiment, the inhibitor binds to factor XI and factor XII.

In one embodiment, the inhibitor of the invention has the property to inhibit factor XI and/or factor XII. In a preferred embodiment, the inhibitor of the invention has the property to inhibit factor XI and factor XII. In particular, in one embodiment, the inhibitor of the invention has the property to inhibit coagulation activities associated to factor XI and/or XII. In a more preferred embodiment, the inhibitor of the invention has the property to inhibit coagulation activities associated to factor XI and XII.

In one embodiment, the inhibitor of the invention inhibits factor XI activity to at least 30%, at least 40%, at least 50%, at least 60%, at least 65%, at least 70%, at least 75%, at least 80%, or to at least 85%.

In another embodiment, the inhibitor of the invention inhibits factor XII activity to at least 30%, at least 40%, at least 50%, at least 60%, at least 65%, at least 70%, at least 75%, at least 80%, or to at least 85%.

In another embodiment, the inhibitor of the invention inhibits factor XI activity to at least 30%, 40%, 50%, 60%, 65%, 70%, 75%, 80%, or 85%, and inhibits factor XII activity to at least 30%, 40%, 50%, 60%, 65%, 70%, 75%, 80%, or 85%.

Another object of the present invention is the use of an inhibitor of a plasma contact factor as described hereinabove for preventing and/or treating thrombosus.

The present invention further relates to a composition comprising an inhibitor of a plasma contact factor as described hereinabove.

Another object of the invention is a pharmaceutical composition comprising an inhibitor of a plasma contact factor as described hereinabove, and at least one pharmaceutically acceptable excipient.

The present invention also relates to a medicament comprising an inhibitor of a plasma contact factor as described hereinabove, and at least one excipient.

Another object of the invention is a method for preventing and/or treating a plasma contact factor-related disease in a subject in need thereof, comprising administration of an inhibitor of a plasma contact factor. As used herein, the term “preventing” may be replaced by the term “protecting”.

In one embodiment, the method of the invention comprises administration of an inhibitor of a plasma contact factor, wherein said inhibition is selected from the group comprising inhibition of the activation of factor XI into factor XIa by factor XIIa, inhibition of the activation of factor XII into factor XIIa by factor XIa, or a combination thereof.

In one embodiment, the plasma contact factor-related disease of the invention is selected from the group comprising deep vein thrombosis, portal vein thrombosis, jugular vein thrombosis, renal vein thrombosis, pulmonary embolism, unstable angina, acute coronary syndrome, myocardial infraction, cerebral ischemia, and stroke.

In another embodiment, the plasma contact factor-related disease of the invention is the thrombus formation during and/or after the contact of blood with artificial surfaces, such as, for example, stents, intraluminal catheters, valves, percutaneous left ventricular assist pump devices.

In another embodiment, the plasma contact factor-related disease of the invention is the thrombus formation during and/or after a medical procedure such as comprising extracorporeal membrane oxygenation for blood oxygenation, extracorporeal circulation during cardiopulmonary bypass, dialysis and extracorporeal filtration of blood, percutaneous angioplasty, use intraluminal catheters and stents, intra-aortic balloon pump.

In one embodiment, the method of the invention further comprises administration of another compound known to prevent and/or treat a plasma contact factor-related disease.

The present invention further relates to a medical device comprising an inhibitor of a plasma contact factor as described hereinabove.

### EXAMPLES

### Example 1

### Characterisation of the Induced Genes

Genes are induced in the salivary glands of Ixodes ricinus during the slow-feeding phase of the blood meal. The cloning of these genes was carried out by setting up two complementary DNA (cDNA) libraries. The first one is a subtractive library based on the methodology described by Lisitsyn et al. (Science 259, 946-951, 1993) and improved by Diatchenko et al. (Proc. Natl. Acad. Sci. USA 93, 6025-6030, 1996). This library cloned selectively induced mRNA during the tick feeding phase. The second library is a full-length cDNA library, which was constructed by using the basic property of mRNAs (presence of a polyA tail in its 3′end and a cap structure in its 5′ end). This cDNA library permitted the cloning of full-length cDNAs, corresponding to some incomplete cDNA sequences identified in the subtractive cDNA library.

The subtractive library was set up by subtracting uninduced-cDNAs (synthetized from mRNAs equally expressed in the salivary glands of both unfed and engorged ticks) from induced-cDNAs (synthesised from mRNAs differentially expressed in the salivary gland at the end of the slow-feeding phase). The induced-cDNAs was digested by a restriction enzyme, divided into two aliquots, and distinctively modified by the addition of specific adapters. As for the induced-cDNAs, the uninduced cDNAs was also digested by the same restriction enzyme and then mixed in excess to each aliquot of modified induced-cDNA. Each mixture of uninduced-/induced-cDNAs was subjected to a denaturation step, immediately followed by an hybridisation step, leading to a capture of homologous induced-cDNAs by the uninduced-cDNA. Each mixture was then mixed together and subjected again to a new denaturation/hybridisation cycle. Among the hybridised cDNA molecules, the final mixture comprises induced-cDNAs with different adapters at their 5′ and 3′ end. These relevant cDNAs were amplified by polymerase chain reaction (PCR), using primers specific to each adapter located at each end of the cDNA molecules. The PCR products were then ligated into the pCRII™ vector by A-T cloning and cloned in an TOP-10 E. coli strain. The heterogeneity of this subtractive library was evaluated by sequencing 96 randomly chosen recombinant clones. The “induced” property of these cDNA sequences was checked by reverse transcription-PCR (RT-PCR) on mRNA extracted from salivary glands of engorged and unfed ticks. Finally, the full-length induced-cDNA was obtained by screening the full-length cDNA library using, as a probe, some incomplete induced-cDNAs isolated from the subtractive library. These full-length induced DNA molecules were sequenced and compared to known polypeptide and polynucleotide sequences existing in the EMBL/GenBank databases.

The full-length cDNA library was set up by using the strategy developed in the “CapFinder PCR cDNA Library Construction Kit” (Clontech). This library construction kit utilises the unique CapSwitch™ oligonucleotide (patent pending) in the first-strand synthesis, followed by a long-distance PCR amplification to generate high yields of full-length, double-stranded cDNAs. All commonly used cDNA synthesis methods rely on the ability of reverse transcriptase to transcribe mRNA into single stranded DNA in the first-strand reaction. However, because the reverse transcriptase cannot always transcribe the entire mRNA sequence, the 5′ ends of genes tend to be under-represented in cDNA population. This is particularly true for long mRNAs, especially if the first-strand synthesis is primed with oligo(dT) primers only, or if the mRNA has a persistent secondary structure. Furthermore, the use of T4 DNA polymerase to generate blunt cDNA ends after second-strand synthesis commonly results in heterogeneous 5′ ends that are 5-30 nucleotides shorter than the original mRNA. In the CapFinder cDNA synthesis method, a modified oligo(dT) primer is used to prime the first-strand reaction, and the CapSwitch oligonucleotide acts as a short, extended template at the 5′ end for the reverse transcriptase. When the reverse transcriptase reaches the 5′ end of the mRNA, the enzyme switches templates and continues replicating to the end of the CapSwitch oligonucleotide. This switching in most cases occurs at the 7-methylguanosine cap structure, which is present at the 5′ end of all eukaryotic mRNAs. The resulting full-length single stranded cDNA contains the complete 5′ end of the mRNA as well as the sequence complementary to the CapSwitch oligonucleotide, which then serves as a universal PCR priming site (CapSwitch anchor) in the subsequent amplification. The CapSwitch-anchored single stranded cDNA is used directly (without an intervening purification step) for PCR. Only those oligo(dT)-primed single stranded cDNAs having a CapSwitch anchor sequence at the 5′ end can serve as templates and be exponentially amplified using the 3′ and 5′ PCR primers. In most cases, incomplete cDNAs and cDNA transcribed from poly-A RNA will not be recognised by the CapSwitch anchor and therefore will not be amplified.

At the end of these reactions, the full-length cDNA PCR products was ligated into the pCRII cloning vector (Invitrogen) and used for the transformation of XL2 E. coli strain. The full-length cDNA library was then screened by using, as a probe, the incomplete induced-cDNAs isolated from the subtractive library.

Ninety-six clones of subtractive library were randomly sequenced, and their DNA and amino acid translated sequences were compared to DNA and protein present in databases. Among these, 27 distinct family sequences were identified, and 3 of them were selected for further characterisation of their corresponding full-length mRNA sequence. These 3 sequences matched the sequence of i) the human tissue factor pathway inhibitor (TFPI), ii) the human thrombin inhibitor gene, and iii) a snake venom zinc-dependent metalloprotease protein. These genes encode proteins that could be involved in the inhibition of the blood coagulation. The other 24 family sequences presented low or no homologies with polynucleotide and polypeptide sequences existing in databases. Screening of the full-length cDNA library using oligonucleotide probes specific to the 3 previously selected subtractive clones lead to the recovery of the corresponding full-length cDNAs. Random screening of this library led to the selection of 2 other clones. One is closely homologous to an interferon-like protein, whereas the other shows homologies to the Streptococcus equi M protein, an anti-complement protein.

These polypeptides expressed by I. ricinus salivary glands include the polypeptides encoded by the cDNAs defined in the tables, and polypeptides comprising the amino acid sequences which have at least 75% identity to that encoded by the cDNAs defined in the tables over their complete length, and preferable at least 80% identity, and more preferably at least 90% identity. Those with about 95-99% are highly preferred.

The I. ricinus salivary gland polypeptides may be in the form of the “mature” protein or may be a part of a larger protein such as a fusion protein. It may be advantageous to include an additional amino acid sequence, which contains secretory or leader sequences, pro-sequences, sequences which help in purification such as multiple histidine residues, or an additional sequence for stability during recombinant production.

Preferably, all of these polypeptide fragments retain parts of the biological activity (for instance antigenic or immunogenic) of the I. ricinus salivary gland polypeptides, including antigenic activity. Variants of the defined sequence and fragments also form part of the present invention. Preferred variants are those that vary from the referents by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination. Most preferred variants are naturally occurring allelic variants of the I. ricinus salivary gland polypeptide present in I. ricinus salivary glands.

The I. ricinus salivary gland polypeptides of the invention can be prepared in any suitable manner. Such polypeptides include isolated naturally occurring polypeptides, recombinant polypeptides, synthetic polypeptides, or polypeptides produced by a combination of these methods. Means for preparing such polypeptides are well understood in the art.

The I. ricinus salivary gland cDNAs (polynucleotides) include isolated polynucleotides which encode I. ricinus salivary gland polypeptides and fragments thereof, and polynucleotides closely related thereto. More specifically, I. ricinus salivary gland cDNAs of the invention include a polynucleotide comprising the nucleotide sequence of cDNAs defined in the table, encoding an I. ricinus salivary gland polypeptide. The I. ricinus salivary gland cDNAs further include a polynucleotide sequence that has at least 75% identity over its entire length to a nucleotide sequence encoding the I. ricinus salivary gland polypeptide encoded by the cDNAs defined in the tables, and a polynucleotide comprising a nucleotide sequence that is at least 75% identical to that of the cDNAs defined in the tables, in this regard, polynucleotides at least 80% identical are particularly preferred, and those with at least 90% are especially preferred. Furthermore, those with at least 95% are highly preferred and those with at least 98-99% are most highly preferred, with at least 99% being the most preferred. Also included under I. ricinus salivary gland cDNAs is a nucleotide sequence, which has sufficient identity to a nucleotide sequence of a cDNA defined in the tables to hybridise under conditions usable for amplification or for use as a probe or marker. The invention also provides polynucleotides which are complementary to such I. ricinus salivary gland cDNAs.

These nucleotide sequences defined in the tables as a result of the redundancy (degeneracy) of the genetic code may also encode the polypeptides encoded by the genes defined in the tables.

When the polynucleotides of the invention are used for the production of an I. ricinus salivary gland recombinant polypeptide, the polynucleotide may include the coding sequence for the mature polypeptide or a fragment thereof, by itself; the coding sequence for the mature polypeptide or fragment in reading frame with other coding sequences, such as those encoding a leader or secretory sequence, a pre-, or pro- or preproprotein sequence, or other fusion peptide portions. For example, a marker sequence, which facilitates purification of the fused polypeptide can be encoded. Preferably, the marker sequence is a hexa-histidine peptide, as provided in the pQE vector (Qiagen, Inc.) and described in Gentz et al, Proc Natl Acad Sci USA (1989) 86:821-824, or is an HA tag, or is glutathione-s-transferase. The polynucleotide may also contain non-coding 5′ and 3′ sequences, such as transcribed, non-translated sequences, splicing and polyadenylation signals, ribosome binding sites and sequences that stabilize mRNA.

Further preferred embodiments are polynucleotides encoding I. ricinus salivary gland protein variants comprising the amino acid sequence of the I. ricinus salivary gland polypeptide encoded by the cDNAs defined by the table respectively in which several, 10-25, 5-10, 1-5, 1-3, 1-2 or 1 amino acid residues are substituted, deleted or added, in any combination. Most preferred variant polynucleotides are those naturally occurring I. ricinus sequences that encode allelic variants of the I. ricinus salivary gland proteins in I. ricinus.

The present invention further relates to polynucleotides that hybridise preferably stringent conditions to the herein above-described sequences. As herein used, the term “stringent conditions” means hybridisation will occur only if there is at least 80%, and preferably at least 90%, and more preferably at least 95%, yet even more preferably 97-99% identity between the sequences.

Polynucleotides of the invention, which are identical or sufficiently identical to a nucleotide sequence of any gene defined in the table or a fragment thereof, may be used as hybridisation probes for cDNA clones encoding I. ricinus salivary gland polypeptides respectively and to isolate cDNA clones of other genes (including cDNAs encoding homologs and orthologs from species other than I. ricinus) that have a high sequence similarity to the I. ricinus salivary gland cDNAs. Such hybridisation techniques are known to those of skill in the art. Typically these nucleotide sequences are 80% identical, preferably 90% identical, more preferably 95% identical to that of the referent. The probes generally comprise at least 15 nucleotides, preferably, at least 30 nucleotides or at least 50 nucleotides. Particularly preferred probes range between 30 and 50 nucleotides. In one embodiment, to obtain a polynucleotide encoding I. ricinus salivary gland polypeptide, including homologues and orthologues from species other than I. ricinus, comprises the steps of screening an appropriate library under stringent hybridisation conditions with a labelled probe having a nucleotide sequence contained in one of the gene sequences defined by the table, or a fragment thereof; and isolating full-length cDNA clones containing said polynucleotide sequence. Thus in another aspect, I. ricinus salivary gland polynucleotides of the present invention further include a nucleotide sequence comprising a nucleotide sequence that hybridise under stringent condition to a nucleotide sequence having a nucleotide sequence contained in the cDNAs defined in the tables or a fragment thereof. Also included with I. ricinus salivary gland polypeptides are polypeptides comprising amino acid sequences encoded by nucleotide sequences obtained by the above hybridisation conditions (conditions under overnight incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 microgram/ml denatured, sheared salmon sperm DNA, followed by washing the filters in 0.1×SSC at about 65° C.).

The polynucleotides and polypeptides of the present invention may be employed as research reagents and materials for the development of treatments and diagnostics tools specific to animal and human disease.

This invention also relates to the use of I. ricinus salivary gland polypeptides, or I. ricinus salivary gland polynucleotides, for use as diagnostic reagents.

Materials for diagnosis may be obtained from a subject's cells, such as from blood, urine, saliva, tissue biopsy.

Thus in another aspect, the present invention relates to a diagnostic kit for a disease or susceptibility to a disease which comprises:

(a) an I. ricinus salivary gland polynucleotide, preferably the nucleotide sequence of one of the gene sequences defined by the table, or a fragment thereof;
(b) a nucleotide sequence complementary to that of (a);
(c) an I. ricinus salivary gland polypeptide, preferably the polypeptide encoded by one of the gene sequences defined in the table, or a fragment thereof;
(d) an antibody to an I. ricinus salivary gland polypeptide, preferably to the polypeptide encoded by one of the gene sequences defined in the table; or
(e) a phage displaying an antibody to an I. ricinus salivary gland polypeptide, preferably to the polypeptide encoded by one of the cDNAs sequences defined in the table.

It will be appreciated that in any such kit, (a), (b), (c), (d) or (e) may comprise a substantial component.

Another aspect of the invention relates to a method for inducing an immunological response in a mammal which comprises inoculating the mammal with I. ricinus salivary gland polypeptide or epitope-bearing fragments, analogues, outer-membrane vesicles or cells (attenuated or otherwise), adequate to produce antibody and/or T cell immune response to protect said animal from bacteria and viruses which could be transmitted during the blood meal of I. ricinus and related species. In particular the invention relates to the use of I. ricinus salivary gland polypeptides encoded by the cDNAs defined in the tables. Yet another aspect of the invention relates to a method of inducing immunological response in a mammal which comprises, delivering I. ricinus salivary gland polypeptide via a recombinant vector directing expression of I. ricinus salivary gland polynucleotide in vivo in order to induce such an immunological response to produce antibody to protect said animal from diseases transmitted by I. ricinus ticks or other related species (Lyme disease, tick encephalitis virus disease, . . . ).

A further aspect of the invention relates to an immunological composition or vaccine formulation which, when introduced into a mammalian host, induces an immunological response in that mammal to a I. ricinus salivary gland polypeptide wherein the composition comprises a I. ricinus salivary gland cDNA, or I. ricinus salivary gland polypeptide or epitope-bearing fragments, analogs, outer-membrane vesicles or cells (attenuated or otherwise). The vaccine formulation may further comprise a suitable carrier. The I. ricinus salivary gland polypeptide vaccine composition is preferably administered orally or parenterally (including subcutaneous, intramuscular, intravenous, intradermal injection). Formulations suitable for parenteral administration include aqueous and non-aqueous sterile injection solutions which may contain anti-oxidants, buffers, bacteriostats and solutes which render the formulation iotonic with the blood of the recipient; and aqueous and non-aqueous sterile suspensions which may include suspending agents or thickening agents. The formulations may be presented in unit-dose or multi-dose containers, for example; sealed ampoules and vials and may be stored in a freeze-dried condition requiring only the addition of the sterile liquid carrier immediately prior to use. The vaccine formulation may also include adjuvant systems for enhancing the immunogenicity to the formulation, such as oil-in water systems and other systems known in the art. The dosage will depend on the specific activity of the vaccine and can be readily determined by routine experimentation.

Yet another aspect relates to an immunological/vaccine formulation which comprises the polynucleotide of the invention. Such techniques are known in the art, see for example Wolff et al, Sciences, (1990) 247: 1465-8.

Another aspect of the invention related to the use of these I. ricinus salivary gland polypeptides as therapeutic agents. In considering the particular potential therapeutic areas for such products, the fields covered by these products are: haematology (particularly coagulation clinics), transplantation (for immunosuppression control), rheumatology (for anti-inflammatories), and general treatment (for specific or improved anaesthetics).

TABLE 1
Sequences identified in the subtractive and the cDNA full-length libraries

| | Motifs | Similar sequences in databases | Score | Class |
|---|---|---|---|---|
| Seq. 1 | | No significative identity | | III |
| Seq. 2 | | No significative identity | | III |
| Seq. 3 | | No significative identity | | III |
| Seq. 4 | | No significative identity | | III |
| Seq. 5 | Prokariotic mbre lipoprotein lipid attachment site | No significative identity | | III |
| Seq. 6 | | R. melioti Nitrogen fixation (fixF) | 0.00089 | III |
| | | Human Apolipoprotein B-100 | 0.0045 | III |
| | | Hu. mRNA for cAMP response element (CRE-BP1) | 0.057 | III |
| | | binding prot | | |
| Seq. 7 | Kunitz family of serine protease inhibitor | Human BAC clone GS345D13 | 4.7¹³ | I |
| | | H. sap Tissue factor Pathway Inhibitor | 4⁻¹² | I |
| Seq. 8 | | Amino acid sequence of Seq. 7 | | |
| Seq. 9 | Prokariotic membrane lipoprotein lipid | No significative identity | | III |
| | attachment site | | | |
| Seq. 10 | | Pea mRNA for GTP binding protection. | 0.48 | III |
| Seq. 11 | | No significative identity | | III |
| Seq. 12 | | IL-11 R-Beta gene | 0.18 | II |
| Seq. 13 | | No significative identity | | III |
| Seq. 14 | | C. gloeosporioides cutinase gene | 0.082 | III |
| Seq. 15 | | No significative identity | | III |
| Seq. 16 | | Mouse mRNA for secretory protection cont. | 0.014 | III |
| | | thrombospondin motifs | | |
| Seq. 17 | Zinc dependent metallopeptidase family | B. jararaca mRNA for jararhagin | 1.1⁻⁵ | I |
| | | Agkistrodon contortrix metalloproteinase precursor | 3.9⁻⁵ | I |
| Seq. 18 | | Amino acid sequence of Seq. 17 | | |
| Seq. 19 | | O. aries gene for ovine Interferon-alpha | 0.7 | II |
| | | Interferon-omega 45 | 0.88 | II |
| | | Interferon-omega 20 | 0.89 | II |
| | | RCPT PGE2 | 0.85 | III |
| | | PGE Rcpt EP2 | 0.85 | III |
| Seq. 20 | | No significative identity | | III |
| Seq. 21 | | IgG1L chain directed against human IL2 rcpt Tac protein | 0.19 | II |
| | | Var region of light chain of MAK447/179 | 0.2 | II |
| Seq. 22 | | No significative identity | | III |
| Seq. 23 | | No significative identity | | III |
| Seq. 24 | | Mus Musculus neuroactin | 0.42 | III |
| Seq. 25 | | No significative identity | | III |
| Seq. 26 | | H. sapiens thrombin inhibitor | 2.1⁻¹² | I |
| | | Cycloplasmic antiproteinase 38 kDa intracellular serine | 2.3⁻¹² | I |
| | | protection. | | |
| Seq. 27 | | Amino acid sequence of Seq. 26 | | |
| Seq. 28 | | No significative identity | | III |
| Seq. 29 | | No significative identity | | III |
| Seq. 30 | | Mus musculus transcription factor ELF3 (fasta) | 0.053 | III |
| Seq. 31 | | Homo sapiens putative leukocyte interferon-related | 1.70⁻²² | II |
| | | protein (SM15) mRNA | | |
| Seq. 32 | | Amino acid sequence of Seq. 31 | | |
| Seq. 33 | | R. norvegicus mRNA for common antigen-related protein | 4.80⁻⁰⁹ | II |
| Seq. 34 | | Amino acid sequence of Seq. 33 | | |

SEQ. ID. NO. 26 (Iris): homology with H. sapiens thrombin inhibitor 2.1-12, class I
Class I: putative anticoagulant homologs;
Class II: putative immunomodulatory homologs;
Class III: low or no homologies found in the databases).

TABLE 2
Biological characteristics of the selected clones

| | | | | | Signal | | |
|---|---|---|---|---|---|---|---|
| | Full-length sequences similarly to | Fasta/Blastp | ORF | | peptide | Sp length/ | Nucleotide in |
| Clone | databases | Scores^{a} | (aa) | Motifs | scores^{b} | Prob. | position -3^{c} |
| Seq31 | Homo sapiens putative interferon- | 1.8 · 10⁻³⁶/1 · 10⁻⁷¹ | 426 | | D 5.4/F ^{e} | 48aa/8.4 · 10⁻¹ | G |
| | related gene (SKMc15) [U09585] | | | | | | |
| Seq33 | R. norvegicus leukocyte common | 7.8 · 10⁻¹¹/N | 274 | | 10.2/S | 18aa/7.4 · 10⁻³ | A |
| | antigen (LAR) mRNA [X83546] | | | | | | |
| Seq17 | Mouse mRNA for secretory protein | 0.002/6 · 10⁻⁷ | 489 | Metallopeptidase | 7.9/S | 19aa/7.4 · 10⁻⁴ | G |
| | containing thrombospondin motives | | | | | | |
| | [D67076] | | | | | | |
| Seq26 | Pig leukocyte elastase inhibitor | 0/7 · 10⁻⁶⁷ | 378 | Serpin | 8.5/S | 51aa/3.28 · 10⁻³ | A |
| | mRNA [P80229] | | | | | | |
| Seq7 | Human Tissue Factor Pathway | 4.8 · 10⁻¹²/2 · 10⁻⁵ | 87 | Kunitz | 6.5/S | 19aa: 1.8 · 10⁻⁴ | G |
| | Inhibitor [P48307] | | | | | | |

^{a}No score (N)
^{b}Succeeded (S) and Failed (F)
^{c}Guanine (G) and Adenine (A)
^{d} von Heijne analysis
^{e} McGeoch analysis

### Example 2

### Construction of a Representational Difference Analysis (RDA) Subtractive Library

The salivary glands of 5 day engorged or unfed free of pathogen I. ricinus female adult ticks were used in this work.

When removed, these glands were immediately frozen in liquid nitrogen and stored at −80° C. To extract RNA messengers (mRNA), the salivary glands were crushed in liquid nitrogen using a mortar and a pestle. The mRNAs were purified by using an oligo-dT cellulose (Fast Track 2.0 kit, Invitrogen, Groningen, The Netherlands). Two micrograms of mRNAs were extracted from 200 salivary glands of fed ticks, and 1.5 g of mRNAs were also extracted from 1,000 salivary glands of unfed ticks.

All procedures were performed as described by Hubank and Schatz Nucl. Acid Res December 25, vol 22-25 p 5640-5648 (1994). Double-stranded cDNAs were synthesised using the Superscript Choice System (Life Technologies, Rockville, Md. USA). The cDNAs were digested with DpnII restriction enzyme, ligated to R-linkers, amplified with R-24 primers (Hubank and Schatz, 1994), and finally digested again with the same enzyme to generate a “tester” pool consisting of cDNAs from salivary glands of fed ticks and a “driver” pool consisting of cDNAs from salivary glands of unfed ticks. The first round of the subtractive hybridisation process used a tester/driver ratio of 1:100. The second and third rounds utilised a ratio of 1:400 and 1:200,000, respectively. After three cycles of subtraction and amplification, the DpnII-digested differential products were subdivided according to size into 4 different fractions on a 1.7% electrophoresis agarose gel, and subcloned the BamHI site of the pTZ19r cloning vector. The ligated product was used to transform TOP-10 E. coli competent cells (Invitrogen, Groningen, The Nederlands). Nine thousand six hundred clones of this subtractive library were randomly selected, and individually put in 96-well microplates and stored at −80° C. This subtractive library was analysed by sequencing 89 randomly chosen clones, using M13 forward and reverse primers specific to a region located in the pT19r cloning vector. The DNA sequences of these 89 clones were compared, and 27 distinct family sequences were identified. Homology of these sequences to sequences existing in databases is presented in Table 1.

The subtractive sequences 1 to 27 are presented in the sequence-listing file (except for sequences 7, 17 and 26 whose complete mRNA sequences are presented; see also Example 2). Three sequences (SEQ. ID. NO. 7, 17 and 26) were selected for further characterisation of their corresponding full-length mRNA sequence. These 3 sequences matched the sequence of i) the human tissue factor pathway inhibitor (TFPI), ii) a snake venom zinc dependent metallopeptidase protein, and iii) the human thrombin inhibitor protein, corresponding to SEQ. ID. NO. 7, 17 and 26, respectively. These genes encode proteins which could be involved in the inhibition of the blood coagulation or in the modulation of the host immune response.

### Example 3

### Construction of the Full Length cDNA Library and Recovery of Full Length cDNAs Sequences by Screening of this Full Length cDNA Library

This library was set up using mRNAs extracted from salivary glands of engorged ticks. The mRNAs (80 ng) were subjected to reverse transcription using a degenerated oligo-dT primer (5′A(T)30VN-3′), the Smart™ oligonucleotide (Clontech, Palo Alto, USA), and the Superscript II reverse transcriptase (Life Technologies, Rockville, Md., USA). The single strand cDNA mixture was used as template in a hot start PCR assay including the LA Taq polymerase (Takara, Shiga, Japan), the modified oligo-dT primer and a 3′-Smart primer specific to a region located at the 5′ end of the Smart™ oligonucleotide. The PCR protocol applied was: 1 min at 95° C., followed by 25 sec at 95° C./5 min at 68° C., 25 times; and 10 min at 72° C. The amplified double stranded cDNA mixture was purified with a Centricon 30 concentrator (Millipore, Bedford, USA). The cDNAs were divided into 4 fractions ranging from 0.3 to 0.6 kb, 0.6 to 1 kb, 1 kb to 2 kb, and 2 kb to 4 kb on a 0.8% high grade agarose electrophoresis gel. Each fraction was recovered separately by using the Qiaex II extraction kit (Qiagen, Hilden, Germany). The 4 fractions were ligated individually into the pCRII cloning vector included in the TOPO cloning kit (Invitrogen, Groningen, The Netherlands). The ligated fractions were then used to transform XL2-Blue ultracompetent E. coli cells (Stratagene, Heidelburg, Germany). The resulted recombinant clones were stored individually in microplates at −80° C. Ten clones were randomly chosen for partial or complete sequencing. As a result of this procedure, 2 cDNA sequences (SEQ. ID. NO. 31 and SEQ. ID. NO. 33, see Table 1) were selected for their homology to sequence databases. One is closely homologous to an interferon-related protein (SEQ. ID. NO. 31), whereas the other shows homologies to the Rattus norvegicus leukocyte common antigen-related protein (SEQ. ID. NO. 33).

The 4 different fractions of the full-length cDNA library were screened with radiolabelled oligonucleotide probes specific to selected clones identified in the subtractive cDNA library. The labelling of these oligo probes was performed as described in “Current Protocols in Molecular Biology” (Ausubel et al, 1995, J. Wiley and sons, Eds). These 4 different fractions were then plated on nitrocellulose membranes and grown overnight at 37° C. These membranes were denatured in NaOH 0.2M/NaCl 1.5M, neutralised in Tris 0.5M pH 7.5-NaCl 1.5M and fixed in 2×SSC (NaCl 0.3 M/Citric Acid Trisodium di-hydrated 0.03 M). The membranes were heated for 90 min at 80° C., incubated in a pre-hybridisation solution (SSC 6×, Denhardt's 10×, SDS 0.1%) at 55° C. for 90 min., and finally put overnight in a preheated hybridisation solution containing a specific radiolabelled oligonucleotide probe at 55° C. The hybridised membranes were washed 3 times in a SSC 6× solution at 55° C. for 10 min, dried and exposed on Kodak X-OMAT film overnight at −80° C.

The full-length cDNA library was also analysed by sequencing a set of clones. The resulted DNA sequences were compared to EMBL/GenBank databases and were used to set up oligonucleotide probes to recover other corresponding clones. In this way, the complete consensus mRNA sequence of the SEQ. ID. NO. 28 and 29 was confirmed by the recovery of two other clones corresponding to these sequences. Only one full-length cDNA clone corresponding to the subtractive clone 17 was isolated. Therefore, to identify the complete sequence of the SEQ. ID. NO. 17 and SEQ. ID. NO. 26, the Rapid Amplification of cDNA Ends (RACE) method was applied.

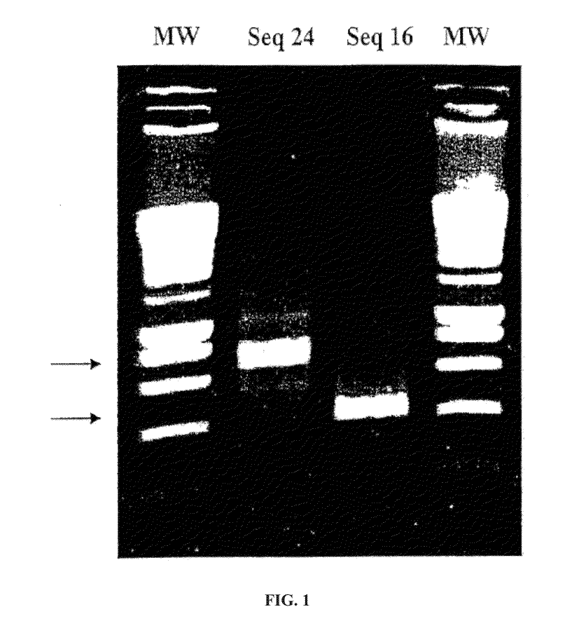

The RACE methodology was performed as described by Frohman et al. Rapid amplification of CDNA Cold Spring Harbor Laboratory press, Cold Spring Harbor, N.Y. p 381-409 (Dieffen bock et al eds) (1995). The reverse transcription step was carried out using 10 ng of mRNAs extracted from salivary glands of engorged ticks and the Thermoscript Reverse transcriptase (Life technologies, Rockville, Md., USA). All gene specific primers (GSP) had an 18 base length with a 61% G/C ratio. The amplified products were subjected to an agarose gel electrophoresis and recovered by using an isotachophorese procedure. The cDNAs were cloned into the pCRII-TOPO cloning vector (Invitrogen, Groningen, The Netherlands). To identify the consensus cDNA sequence, different clones were sequenced, and their sequence was compared to their known corresponding sequence. Therefore, the complete cDNA sequences of the clones 17 and 26 isolated in the subtractive library were obtained by this RACE procedure (FIG. 1).

### Example 4

### Analysis of the Full Sequences of 5 Selected Clones

The sequences of selected clones (SEQ. ID. NO. 7, 17, 26, 31 and 33) allowed identification of the open reading frames, from which the amino sequences were deduced. These potential translation products have a size between 87 and 489 amino acids (see table 2). In order to evaluate, in silico, their respective properties, the amino acid sequences and the nucleotide sequences of said 5 open frames were compared with the databases using the tFasta and Blastp algorithms.

These comparisons show that SEQ. ID. NO. 7 is highly homologous to the human Tissue Factor Pathway Inhibitor (TFPI). TFPI is an inhibitor of serine proteases having 3 tandemly arranged Kunitz-type-protease-inhibitor (KPI) domains. Each of these units or motifs has a particular affinity for different types of proteases. The first and second KPI domains are responsible for the respective inhibition of VIIa and Xa coagulation factors. The third KPI domain apparently has no inhibitory activity. It should be noted that the corresponding polypeptide sequence of SEQ. ID. NO. 7 cDNA clone is homologous to the region of the first KPI domain of TFPI and that the KPI is perfectly kept therein. This similarity suggests that the SEQ. ID. NO. 7 protein is a potential factor VIIa inhibitor.

The amino sequence deduced from the SEQ. ID. NO. 28 clone has a great homology with 3 database sequences, namely: mouse TIS7 protein, rat PC4 protein and human SKMc15 protein. These 3 proteins are described as putative interferon type factors. They possess very well conserved regions of the B2 interferon protein. Therefore, it is proposed that the SEQ. ID. NO. 31 protein has advantageous immunomodulatory properties.

Sequences SEQ. ID. NO. 17 and SEQ. ID. NO. 26 were compared with databases showing homology with the Gloydius halys (sub-order of ophidians) M12b metallopeptidase and the porcine elastase inhibitor belonging to the super-family of the serine protease inhibitors (Serpin), respectively. The amino sequences of these 2 clones also have specific motifs of said families. It is proposed that said proteins have advantageous anticoagulant and immuno-modulatory properties.

Finally, the SEQ. ID. NO. 33 clone has a weak homology with the R. norvegicus leukocyte common antigen (LAR) that is an adhesion molecule. It is thus possible that the SEQ. ID. NO. 33 protein has immunomodulatory properties related to those expressed by the LAR protein.

Due to their potential properties, most of the proteins examined are expected to be secreted in the tick saliva during the blood meal. Accordingly, tests were made for finding the presence of a signal peptide at the beginning of the deduced amino sequences. By the McGeoch method (Virus Res 3: 271-286, 1985), signal peptide sequences were detected for the SEQ. ID. NO. 7, SEQ. ID. NO. 17, SEQ. ID. NO. 26 and SEQ. ID. NO. 33 deduced amino sequences. It seems that said proteins are secreted in the tick salivary gland. Furthermore, the presence of a Kozak consensus sequence was observed upstream of the coding sequences of all examined clones. This indicates that their mRNAs potentially could be translated to proteins.

### Example 5

### Evaluation of the Differential Expression of the cDNA Clones Isolated in the Subtractive and Full-Length cDNA Libraries

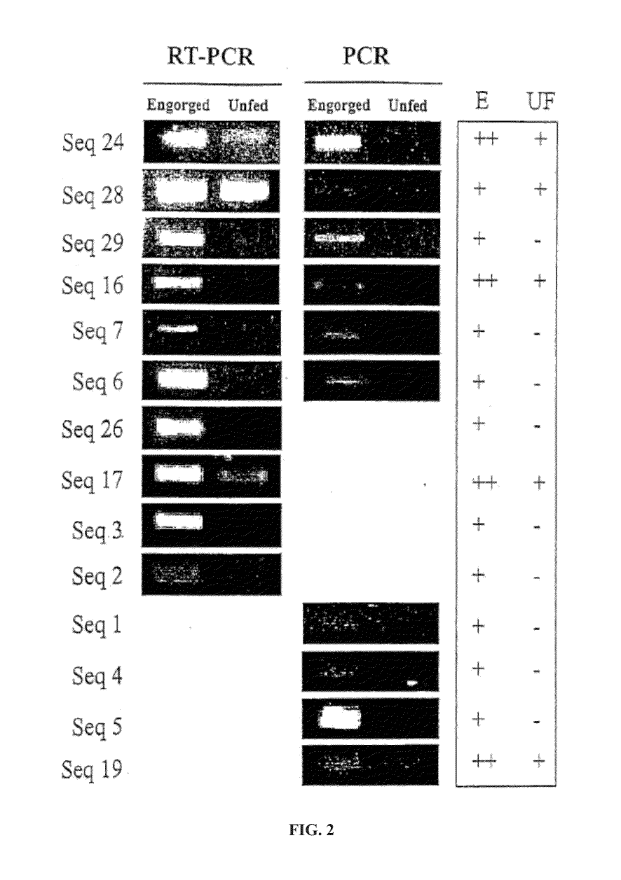

The differential expression of the mRNAs corresponding to the 5 full-length selected clones (SEQ. ID. NO. 7, SEQ. ID. NO. 17, SEQ. ID. NO. 26, SEQ. ID. NO. 31 and SEQ. ID. NO. 33) and of 9 subtractive clones was assessed using a PCR and a RT-PCR assays (FIG. 2).

The PCR assays were carried out using as DNA template cDNAs obtained from a reverse transcription procedure on mRNAs extracted from salivary glands either of engorged or of unfed ticks.

Each PCR assay included pair of primers specific to each target subtractive or cDNAs full-length sequence. PCR assays were performed in a final volume of 50 μl containing 20 pM primers, 0.2 mM deoxynucleotide (dATP, dCTP, dGTP and dTTP; Boehringer Mannheim GmbH, Mannheim, Germany), PCR buffer (10 mM TrisHCl, 50 mM KCI, 2.5 mM. MgCl₂, pH 8.3) and 2.5 U of Taq DNA polymerase (Boehringer Mannheim GmbH, Mannheim, Germany).

DNA samples were amplified for 35 cycles under the following conditions: 94 C for 1 min., 72 C for 1 min. and 64 C for 1 min, followed by a final elongation step of 72 C for 7 min.

The RT-PCR assay was carried out on the 5 selected full-length cDNA clones and on 5 cDNA subtractive clones. The mRNAs used as template in the reverse transcription assay was extracted from salivary glands of engorged and unfed I. ricinus ticks. The reverse transcription assays were performed using a specific primer (that target one the selected sequences) and the “Thermoscript Reverse transcriptase” (Life technologies, Rockville, Md., USA) at 60° C. for 50 min. Each PCR assay utilised the reverse transcription specific primer and an another specific primer. The PCR assays were performed in a final volume of 50 μl containing 1 μM primers, 0.2 mM deoxynucleotide (dATP, dCTP, dGTP and dTTP; Boehringer Mannheim GmbH, Mannheim, Germany), PCR buffer (10 mM Tris HCI, 50 mM KCl, 2.5 mM MgCl₂, pH 8.3) and 2.5 U of Expand High Fidelity polymerase (Roche, Bruxelles, Belgium). Single stranded DNA samples were amplified for 30 cycles under the following conditions: 95° C. for 1 min., 72° C. for 30 sec. and 60° C. for 1 min, followed by a final elongation step of 72° C. for 7 min.

The FIG. 2 shows that the expression of the selected sequences is induced in salivary glands of 5 day engorged ticks, except for the sequence 31 that is expressed at a similar level in salivary glands of engorged and unfed ticks. The expression of the other mRNAs could be either induced specifically or increased during the blood meal.

### Example 6

### Expression of Recombinant Proteins in Mammal Cells

The study of the properties of isolated sequences involves the expression thereof in expression systems allowing large amounts of proteins encoded by these sequences to be produced and purified.

The DNA sequences of the 5 selected clones (SEQ. ID. NO. 7, SEQ. ID. NO. 17, SEQ. ID. NO. 26, SEQ. ID. NO. 31 and SEQ. ID. NO. 33) were transferred into the pCDNA3.1 His/V5 expression vector. Said vector allows the expression of heterologous proteins fused to a tail of 6 histidines as well as to the V5 epitope in eucaryotic cells. The different DNAs were produced by RT-PCR by using primers specific to the corresponding clones. These primers were constructed so as to remove the stop codon of each open reading frame or phase in order to allow the protein to be fused to the 6×HIS/Epitope V5 tail. In addition, the primers contained restriction sites adapted to the cloning in the expression vector. Care was taken to use, when amplifying, a high fidelity DNA polymerase (Pfu polymerase, Promega).

The transient expression of the Seq16 and 24 recombinant proteins was measured after transfection of the Seq16 and Seq24-pCDNA3.1-His/V5 constructions in COS1 cells, using Fugen 6 (Boehringer). The protein extracts of the culture media corresponding to times 24, 48 and 72 hours after transfection were analysed on acrylamide gel by staining with Coomassie blue or by Western blot using on the one hand an anti-6× histidine antibody or on the other hand Nickel chelate beads coupled to alcaline phosphatase.

These analyses showed the expression of said proteins in the cell culture media.

### Example 7

### Expression of Proteins in E. coli

7.1. Insertion of Coding Sequences into the pMAL-C2E Expression Vector.

Proteins fused with the Maltose-Binding-Protein (MBP) were expressed in bacteria by using the pMAL-C2E (NEB) vector. The protein of interest then could be separated from the MBP thanks to a site separating the MBP from the protein, said site being specific to protease enterokinase.

In order to express optimally the 5 sequences examined, using the pMAL-C2E vector, PCR primer pairs complementary to 20 bases located upstream of the STOP codon and to 20 bases located downstream of the ATG of the open reading frame or phase were constructed. The amplified cDNA fragments only comprise the coding sequence of the target mRNA provided with its stop codon. The protein of interest was fused to MBP by its N-terminal end. On the other hand, since these primers contained specific restriction sites specific to the expression vector, it was possible to effect direct cloning of the cDNAs. The use of Pfu DNA polymerase (Promega) made it possible to amplify the cDNAs without having to fear for errors introduced into the amplified sequences.

The coding sequences of clones SEQ. ID. NO. 7, SEQ. ID. NO. 17, SEQ. ID. NO. 26 and SEQ. ID. NO. 31 were reconstructed in that way. Competent TG1 cells of E. coli were transformed using these constructions. Enzymatic digestions of these mini-preparations of plasmidic DNA made it possible to check that the majority of clones SEQ. ID. NO. 7, SEQ. ID. NO. 17, SEQ. ID. NO. 26 and 31-p-MALC2-E effectively were recombinant.

### 7.2. Expression of Recombinant Proteins.

Starting from various constructions cloned in TG1 E. coli cells, the study of the expression of recombinant proteins fused with MBP was initiated for all sequences of interest (i.e. SEQ. ID. NO. 7, SEQ. ID. NO. 17, SEQ. ID. NO. 26 and SEQ. ID. NO. 33) except for SEQ. ID. NO. 31. The culture of representative clones of SEQ. ID. NO. 7, SEQ. ID. NO. 17, SEQ. ID. NO. 26 and SEQ. ID. NO. 33 as well as negative controls (non recombinant plasmids) were started to induce the expression of recombinant proteins therein. These cultures were centrifuged and the pellets were separated from the media for being suspended in 15 mM pH7.5 Tris and passed through the French press. The analysis of these samples on 10% acrylamide gel coloured with Coomassie blue or by Western Blot using rabbit anti-MBP antibodies, showed the expression of recombinant proteins SEQ. ID. NO. 7 (˜50 kDa), SEQ. ID. NO. 17 (˜92 kDA), SEQ. ID. NO. 26 (˜80 kDA) and SEQ. ID. NO. 31 (−67 kDa).

### Example 8

### Production of Antibodies

The SEQ. ID. NO. 7, SEQ. ID. NO. 17 and SEQ. ID. NO. 26 protein were injected into groups of 4 mice with the purpose of producing antibodies directed against said proteins. The antigens were firstly injected with the complete Freund adjuvant. Two weeks later, a recall injection was made with incomplete Freund adjuvant. The sera of mice injected with SEQ. ID. NO. 17 provided positive tests for anti-MBP antibodies.

### Example 9

### Ir-CPI (Sequence SEQ. ID. NO. 7 without its Peptide Signal) Characteristics

### Ticks and Tick Salivary Gland Extracts.

I. ricinus ticks were bred and maintained at the Institute of Zoology, University of Neuchatel (Switzerland). Colony founders were initially collected in the field near Neuchatel and have been maintained on rabbits and mice for over 20 years. Pairs of adult (female and male) ticks were allowed to anchor and feed on rabbits. For preparation of salivary gland extracts (SGE), Five-day engorged female ticks were dissected under the microscope. Salivary glands were harvested in ice cold phosphate saline buffer. Tissues were then disrupted and homogenized using a dounce. Samples were centrifuged for 5 minutes at 10,000 g and the supernatants were recovered and stored at −20° C.

Expression and Purification of Recombinant Ir-CPI in E. coli.

The coding region of Ir-CPI cDNA was amplified using a forward primer corresponding to the predicted N-terminal end of mature Ir-CPI (5′-CGCGGATCCGCGGCCAACCACAAAGGTAGAGGG-3′) and a reverse primer (5′-CCGCTCGAGCGGTTAGACTTTTTTTGCTCTGCATTCC-3′) corresponding to the C-terminal end of Ir-CPI including the stop codon. BamHI and XhoI restriction enzyme digestion sites were engineered into the 5′ and 3′ primers, respectively, to enable cloning into the pGEX-6P-1 expression vector (GE Healthcare, Sweden). PCR were performed in a 50 μl reaction volume containing 2.5 U of Taq polymerase (Takara Ex Taq, Takara, Japan), 10 pmoles of specific primers and 2.5 nmoles of each dNTP (Takara) in a standard buffer supplied by the manufacturer (Takara). PCR conditions were as follows: 1 cycle at 95° C. for 4 min followed by 30 cycles at 95° C. for 30 s/58° C. for 30 s/72° C. for 30 s followed by a final extension step at 72° C. for 10 min. PCR products were then purified by polyacrylamide gel electrophoresis followed by electroelution. The PCR product was cloned in-frame with GST in the pGEX-6P-1 vector at the BamHI and EcoRI restriction sites and transformed into E. coli strain BL21. Production of the recombinant protein was induced by the addition of IPTG at a final concentration of 1 mM and shaking at 37° C. for 2 h. Bacteria were harvested by centrifugation at 4000 g for 20 min and the pellet was dissolved in PBS. Lysates containing the expressed fusion protein were prepared using a French press. The resulting supernatant, which contained the GST-Ir-CPI fusion protein, was incubated with Glutathione Sepharose High Performance (GE Healthcare, Sweden) and washed. Ir-CPI was released by cleaving with PreScission protease according to the manufacturer's specifications and then purified to homogeneity by gel filtration chromatography using a HiLoad Superdex 75 column (GE Healthcare, Sweden).

### Primary Hemostasis.

Human blood samples were collected from healthy donors in 3.8% trisodium citrate tubes. Global platelet function was measured on a PFA-100 machine (Dade Berhing) with collagen/epinephrine or collagen/ADP cartridge. The sample ( 1/10 protein in HBSS and 9/10 citrated whole blood) was aspirated through a capillary under steady high shear rates within 45 min of sample collection. A platelet plug was formed because of presence of the platelet agonist and the high shear rates, and this gradually occluded the aperture. The closure time was considered to be the time required to obtain full occlusion of the aperture.

### Anticoagulant Activity.

The anticoagulant activities of Ir-CPI (presenting the sequence SEQ. ID. NO. 7 without its peptide signal) were determined by four coagulation tests using a Start8 coagulometer. Human blood samples were collected from healthy donors in 3.8% trisodium citrate, and platelet-poor plasma was obtained by further centrifugation at 4000 g for 10 min.

Activated Partial Thromboplastin Time (aPTT)—

Plasma (25 μl) and Ir-CPI (25 μl) were preincubated for 2 min at 37° C. Mixtures were activated for 4 min with 25 μl of Actin FS® (Dade Berhing, Germany). Clotting was initiated by adding 50 μl of 25 mM CaCl₂.

### Prothrombin Time (PT)—

Plasma (25 μl) and Ir-CPI (25 μl) were preincubated for 2 min at 37° C. Mixtures were activated for 4 min with 25 μl of Innovin® 1/200 (Dade Berhing, Germany). The clotting reaction was started by adding 50 μl of 25 mM CaCl₂.

### Stypven Time—

Plasma (25 μl), Hepes buffer (50 μl—Hepes 25 mM, Glycine 2%, NaCl 145 mM; pH 7.35) and Ir-CPI (25 μl) were preincubated for 2 min at 37° C. Clotting was initiated by the addition of 25 μl of LA 1 (Diagnostica Stago).

### Thrombin Time—

Plasma (25 μl), Hepes buffer (50 μl) and Ir-CPI (25 μl) were preincubated for 2 min at 37° C. Clotting was initiated by the addition of 25 μl of Thrombin (Diagnostica Stago).

### Determination of Clot Lysis Times.

Clot lysis times on platelet-poor plasma were determined as described by Zouaoui Boudjeltia et al. (BMC Biotechnol. 2, 2:8, 2002). Plasma (100 μl), t-PA (25 μl) and Ir-CPI (100 μl) were preincubated for 2 min at 37° C. Clot formation was started by adding 100 μl (1.5 U/ml) of thrombin. The clot lysis time was measured with a semi-automatic instrument.

### Assay of Alternative Pathway (AP) and Classical Pathway (CP) Complement Activity

The capacity of Ir-CPI to inhibit the alternative complement pathway (AP) was determined according to Giclas PC (1997) Complement tests. In: Rose N R, Conway de Macario E, Folds J D, Lane H C & Nakamura R M, editors. Manual of clinical laboratory immunology, 5th edition, ASM Press, Washington D.C. pp. 181-186, on red blood cells (RBC) from naïve healthy female New Zealand White rabbits. Briefly, fresh sera were diluted in gelatin-veronal-EGTA buffer (GVB) in microwell plates and washed RBCs were added. After 60 min of incubation at 37° C., supernatants were recovered to measure absorbance at 415 nm with a Model 680 microplate reader (Biorad). The volume of serum causing 50% hemolysis (AHSO value) was then determined by serial dilutions and used for further tests. The 100% lysis control was the total hemolysis produced by incubating 25 μl of MilliQ water. Background level (no hemolysis) was determined by incubating the erythrocytes in GVB buffer alone (without added serum). In order to test the inhibitory effect of Ir-CPI, 10 μg were introduced in the AP test. Ir-CPI was serially diluted in a final volume of 25 μl GVB in the presence of AHSO volume of the host serum under consideration. The assay then proceeded as described above. Percent inhibition of hemolysis was calculated as follows: (OD₄₁₅ₙₘ [serum+inhibitor]−OD₄₁₅ₙₘ GVB control/OD₄₁₅ₙₘ [serum only]−OD₄₁₅ₙₘ GVB control)×100.

The capacity of Ir-CPI to inhibit the classical complement pathway (CP) was also determined essentially as described by Colligan J E (1994) Complement. In: Coligan J E, Kruisbeek A M, Margulies D H, Shevach E M, Strober W, editors. Current Protocols in Immunology. Wiley/Interscience, New-York. pp. 13.1.1-13.2.7. Ready-to-use reagents were purchased from Institut Virion\Serion GmbH (Wurtzburg, Germany). They included sheep erythrocytes pre-coated with rabbit anti-sheep RBC antibodies and Veronal Buffer pH 7.3 (VB) containing NaCl, CaCl₂ and MgCl₂. Briefly, diluted serum was incubated in the presence of antibody-coated sheep RBCs in microplates. Pooled human serum was first titrated to determine the volume that produces 50% hemolysis (CH50 value). Two-fold dilutions of Ir-CPI starting with 10 μg were prepared in VB buffer containing the equivalent of 0.8 μl human serum per test (total volume 25 μl). Pre-coated sheep erythrocytes were then added and the reaction performed as described above. Results were expressed as percent inhibition of hemolysis in the same was as for the AP pathway.

### Thrombin Activity Profiles.

Materials. PPP reagent (5 pM TF and 4 μM PL in the final mixture), PPP LOW reagent (1 pM TF and 4 μM PL in the final mixture) and thrombin calibrator were purchased from Synapse BV. For each experiment, a fresh mixture of fluorogenic substrate/calcium chloride buffer solution was prepared as follows: 2275 μl of buffer (Hepes 20 mM, pH 7.35) containing 60 mg/ml of bovine serum albumin (Sigma) and 240 μl of 1 M calcium chloride were mixed with 60 μl of 100 mM DMSO solution of fluorogenic thrombin substrate (Z-Gly-Gly-Arg-AMC, Bachem). Actin FS® was obtained from Dade-Behring and was diluted 25 fold with distilled water.

Preparation of human plasma. Blood from male healthy volunteers, who were free from medication for at least two weeks, was taken by venipuncture and collected into 0.105 M sodium citrate (9:1 vol/vol). Platelet-poor plasma (PPP) was obtained by centrifugation at room temperature for 10 minutes at 2,500 g and was used immediately after centrifugation.

Calibrated automated thrombin activity measurement. Thrombin activity measurement was performed using the previously reported CAT procedure (Hemker et al. Pathophysiol. Haemost Thtomb. 2003, vol 33 (1) p 4-15). Briefly, 80 μl of PPP, 10 μl of PBS or Ir-CPI and 20 μl of PPP reagent, PPP LOW reagent or diluted Actin FS were mixed in a 96-wells microtiter plate (Thermo Immulon 2HB) and were incubated for 5 minutes at 37° C. The coagulation process was triggered by addition of 20 μl of substrate/calcium chloride buffer at 37° C. A calibration condition was also realized. In this later case, the same protocol as described above using PBS was followed but the activator was replaced by 20 μl of thrombin calibrator. The reaction of fluorogenic thrombin substrate hydrolysis was monitored on a microplate fluorometer Fluoroskan Ascent FL (Thermo Labsystems) with a 390/460 nm filter set (excitation/emission). Fluorescence was measured every 20 s for 60 min. The commercially available Thrombinoscope® software (Synapse BV) processed automatically the acquired data to give thrombin activity profile curves and measurement parameters (lag time and Cmax). Ten Ir-CPI concentrations ranging from 0.001 to 9,077 μM were tested in each experiment in triplicate.

Design of Small Interference RNA (siRNA).

Three siRNA were designed to target Ir-CPI mRNA and were synthesized by Eurogentec. These were 5′-CCAUGCAGAGCACGAAUUC-3′, 5′-GCACGAAUUCCGAGUUACU-3′ and 5′-ACUACGUGCCAAGAGGAAU-3′, respectively.

Ex Vivo Incubation of siRNA with Salivary Gland Extracts.

The salivary glands from 10 partially (5 days) fed female ticks were incubated for 6 h at 37° C. in the presence of 5 μg of siRNA negative control duplexes (Eurogentec, Belgium) or Ir-CPI siRNA or buffer alone in a total volume of 200 μl of incubation buffer TC-199 (Sigma) containing 20 mM MOPS, pH 7.0.

### RT-PCR Analysis to Confirm Gene Silencing.

Messenger RNA from salivary gland extracts was isolated by oligo-dT chromatography (MicroFastTrack 2.0 mRNA Isolation Kit, Invitrogen). Reverse transcription was routinely performed in a 20 μl standard RT reaction mixture according to the manufacturer's instructions (First-Strand cDNA Synthesis System, Invitrogen) using the oligo dT primer. PCR was routinely performed in 50 μl of standard Takara buffer containing 2.5 U of Taq polymerase (Takara Ex Taq, Takara, Japan), 10 pmoles of each primer, and 2.5 nmoles of each dNTP (Takara). PCR cycling conditions were as follows: 30 cycles of 95° C. 30 s/58° C. 30 s/72° C. 30 s to 1 min 30 s preceded by an initial 4 min denaturation step at 95° C. and followed by a final 10 min extension at 72° C. Primers (sense-primer: 5′-ATGAAACTAACGATGCAGCTGATC-3′ and anti-sense primer: 5-TTAGACTTTTTTTGCTCTGCATTCC-3′) designed to amplify the Ir-CPI open reading frame were used to perform RT-PCR analysis of the transcripts. A pair of primers designed to amplify a 1,131 bp fragment from the actin open reading frame (sense-primer; 5′-ATGTGTGACGACGAGGTTGCC-3′ and anti-sense primer; 5′-TTAGAAGCACTTGCGGTGGATG-3′) were used as controls. 10 μl of the PCR reactions were analyzed on a 2% agarose gel.

Activated Partial Thromboplastin Time (aPTT) and prothrombin Time (PT) assay to confirm gene silencing.

Salivary gland extracts incubated with siRNA were assayed for anticoagulant activity in the aPTT or PT assay. SGE (5 μg) and plasma (25 μl) were preincubated for 2 min at 37° C. Mixtures were activated for 4 min with 25 μl of Actin FS for the aPTT (Dade Berhing) or Innovin 1/200 (Dade Berhing) for the PT. The clotting reaction was started by adding 50 μl of 25 mM CaCl2.

### Assay of the Inhibitory Effect of Ir-CPI on Coagulation Factors.

The inhibitory activity of Ir-CPI was examined on 9 serine proteases: procoagulant serine proteases (plasma kallikrein, FXIIa, FXIa, FIXa, FXa, thrombin and FVIIa) and fibrinolytic serine proteases (t-PA and plasmin). Each serine protease was preincubated with Ir-CPI in a 1:5 molar ratio for 5 min at 37° C., followed by the addition of the appropriate chromogenic substrate (final concentration 0.5 mM). Final concentrations in a total volume of 200 μl in 96-microwell-plates were as follows: kallikrein (3 nM)/S-2302, FXIIa (62.5 nM)/S-2302, FXIa (31.25 nM)/52366, FIXa (500 nM)/Spectrozyme FIXa, FXa (10 nM)/S-2222, Thrombin (35 nM)/Spectrozyme TH, FT-FVIIa (100 nM)/Spectrozyme FVIIa, t-PA (35 nM)/Spectrozyme t-PA, plasmin (30 nM)/Spectrozyme PL. The kinetics of substrate hydrolysis were measured over 3 min Chromogenic substrates S-2302, S-2366 and S-2222 were supplied by Chromogenix AB and Spectrozyme FIXa, TH, FVIIa, t-PA, PL were obtained from American Diagnostica Inc.

### Assay of the Effects of Ir-CPI on Contact System Activation in Plasma.

The effects of Ir-CPI on the activation of the contact system in human plasma were assessed from the generation of activated contact factors (factor XIa, factor XIIa and kallikrein). Human plasma was treated with acid to inactivate plasma serine protease inhibitors and then diluted 1:10 in buffer. Fifty microliters of diluted plasma were incubated with 20 μl of various concentrations of Ir-CPI for 5 min and then activated with 5 μl of aPTT reagent (Actin FS). After 10 min, a chromogenic substrate mixture at a final concentration of 0.5 mM and one or two inhibitors, Corn Trypsine Inhibitor (100 nM) or kallistop (50 μM), were added, and the amidolytic activity of the generated enzyme was determined at 405 nm Sets of added chromogenic substrate and inhibitors were as follows: S-2366, Kallistop and CTI for factor XIa assay; S-2302 and Kallistop for factor XIIa assay; and S-2302 and CTI for kallikrein assay.

### Assay of the Effect of Ir-CPI in a Reconstituted System.

A reconstitution assay of the kallikrein-kininogen-kinin system was performed using purified coagulation factors (FXIIa and prekallikrein). FXIIa (12.5 nM) was preincubated with Ir-CPI in Hepes buffer for 2 min at 37° C. Prekallikrein (12.5 nM) was added to the mixture, and then prekallikrein activation started. After 10 min, chromogenic substrate S-2302 was added, and the increase in absorbance at 405 nm was recorded over 3 min.

Reconstitution assays of the intrinsic coagulation pathway were performed using purified coagulation factors, factor XI/XIa and factor XII/XIIa. The effect of Ir-CPI on the activation of factor XI by factor XIIa was tested by incubating factor XI (15 nM), factor XIIa (60 nM) and Ir-CPI for 10 min at 37° C. After incubation, substrate S-2366 was added and the increase in absorbance was measured. The effect of Ir-CPI on the activation of factor XII by factor XIa was tested by incubating factor XI (15 nM), factor XIIa (60 nM) and Ir-CPI for 10 min at 37° C. After incubation, substrate S-2302 was added and the increase in absorbance was measured.

Reconstitution assays of the extrinsic coagulation pathway were performed using Actichrome TFPI Activity Assay and recombinant human TFPI according to the manufacturer's specifications (American diagnostica, Stamford).

Reconstitution assay of the fibrinolysis system was performed using purified fibrinolytic factors (t-PA and plasminogen). Plasminogen (500 nM) was preincubated with Ir-CPI for 2 min at 37° C. t-PA (500 nM) was added to the mixture, and plasminogen activation started. After 10 mM, Spectrozyme PL chromogenic substrate was added, and the absorbance at 405 nm was measured over 3 min.

### Binding Analysis Using Surface Plasmon Resonance.

The interaction between Ir-CPI and coagulation or fibrinolytic factors was monitored using a BIAcore 3000 instrument (BIAcore AB, Sweden). Ir-CPI (15 μM) was immobilized on the surface of a CM5 sensor chip in 10 mM acetate buffer, pH 5.0, by the amine coupling procedure according to the manufacturer's instructions. 1500 resonance units (RU) of immobilized Ir-CPI were used for the assay. To subtract the non-specific component from the apparent binding response, a blank flow cell was prepared using the same immobilizing procedure without Ir-CPI. Binding analyses were carried out using HBS buffer (HEPES 10 mM, NaCl 150 mM, EDTA 3 mM; pH 7.4 with 0.005% surfactant P20) as running buffer at 25° C. 100 μl of each analyte (100 nM) was injected on the sensor chip at a flow rate of 70 μl/min Association was monitored during an 84 s injection of analyte. Dissociation was monitored for 3 min after return to the running buffer. Regeneration of the sensor chip surface was achieved with a pulse injection (15 μl) of 25 mM NaOH.

The kinetics of interactions between Ir-CPI and the four interacting factors were carried out after a new immobilization of Ir-CPI. The quantity of Ir-CPI immobilized for measurements of kinetics was deliberately maintained at a low level (to approximately 200 RU) to avoid the problems of limitation of the reaction by the process of mass-transport Independence with respect to differences in flow of the initial rate of connection, measured by linear regression at the start of the kinetics after injections of analytes with increasing flows (30 to 70 μl/min) confirmed that the reactions were not limited by such a process. Interaction kinetics were determined, for each analyte, with 6 different concentrations (from 5 nM to 300 nM). Binding data were analyzed using BIA evaluation software to determine the kinetic constants.

### Assay of the Effect of Ir-CPI on Activation of the Classical Complement Pathway by Hageman Factor Fragment (HFf).

The effects of Ir-CPI on activation of the classical complement pathway by Hageman factor fragment (HFf) were assessed using a hemolytic assay. HFf was activated by kallikrein and purified as described by Ghebrehiwet et al. HFf was incubated with various concentrations of Ir-CPI for 5 min. Then 1 μl of human serum and 50 μl of sensitized sheep erythrocytes (EA 10⁸/ml) were added and incubated for 60 min at 37° C. The reaction was stopped by addition of 150 μl of NaCl 0.9%, the mixture was centrifuged, and free hemoglobin was measured in the supernatant at 415 nm.

### Determination of Radioactivity of ¹²⁵I-Ir-CPI in Rat Blood.

¹²⁵I-labeled Ir-CPI was prepared by iodination with [¹²⁵I] sodium iodide in 20 mCi/mg of protein, using IODO-BEADS Iodination Reagent (PIERCE) according to the manufacturer's instructions. Free iodide was removed by extensive gel filtration on Sephadex G10.

The in vivo distribution of ¹²⁵I-Ir-CPI in rat blood was evaluated after i.v. administration. Samples containing 10×10⁶ cpm were resuspended in 200 μl of PBS and administered to rats. Blood was collected after 3, 20, 40 or 60 h by cardiac puncture in 3.8% trisodium citrate. Plasma was obtained by centrifugation, and aliquots of 500 μl were placed in glass test tubes. Radioactivity was determined in a gamma counter.

Ex Vivo Effect of Ir-CPI on aPTT, PT and Fibrinolysis.

The ex vivo effect of Ir-CPI on aPTT, PT and fibrinolysis tests was evaluated using a Start8 coagulometer. Ir-CPI was administered i.v. to rats and blood was collected after 5 min by cardiac puncture in 3.8% trisodium citrate. Platelet-poor plasma was obtained by centrifugation at 4000 g for 10 min. The aPTT, PT and fibrinolysis times were measured using the above-described procedures.

### Bleeding Effect

A rat-tail-transection model was used to evaluate the effect of Ir-CPI on bleeding time. Rats were anesthetized and Ir-CPI was administered i.v. into the vena cava. After 5 min, the rat tail was cut 3 mm from the tip and carefully immersed in 10 ml of distilled water at room temperature. The hemoglobin content of the aqueous solution (absorbance at 540 nm) was used to estimate blood loss. Appropriate controls (i.v. injection of PBS) were run in parallel.

Complete Stasis Combined with Vessel Injury Induced Venous Thrombosis Model in the Rat

Animals. Studies were carried out using male Sprague-Dawley OFA rats weighing 250 to 300 g obtained from Harlan (The Netherlands).

Thrombosis model. Thrombus formation was induced by a combination of complete stasis and vessel injury by ferric chloride according to the modification of the method described by Peternel et al. Thrombosis Research vol 115(6) p 527-534 (2005). Rats were anesthetized with pentobarbital sodium (70 mg/kg, IP). During anesthesia, the abdomen was opened by making an incision along the linea alba towards the sternum, followed by exposition of the posterior vena cava. Surgical threads, 1 cm apart, were placed loosely around the vena cava beneath the renal veins and above the bifurcation of the iliac veins to form a snare. Complete stasis was induced in the posterior vena cava by tightening the downstream snare firmly around the posterior vena cava. Simultaneously, a piece of filter paper (0.3×0.8 cm) saturated with 10% w/v ferric chloride solution was applied to the external surface of the posterior vena cava caudally of the ligature for 10 min Ten min after the removal of the filter paper, the upstream snare was firmly tightened around the posterior vena cava and the rat was then euthanized. The ligated venous segment was excised, the thrombus removed, blotted of excess blood and immediately weighed. Results were expressed in milligrams of thrombus weight by kilograms of rat weight. Ir-CPI (0.5-1000 μg/kg) or saline were injected in the left femoral vein 5 min prior to the induction of the thrombus formation.

### Results

### Protein Properties—Expression and Purification of Recombinant Ir-CPI.

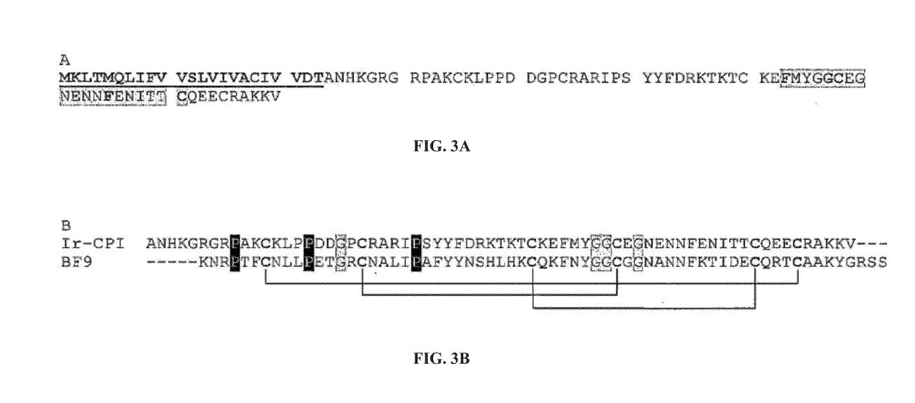

To identify cDNAs encoding proteins specifically expressed during the blood meal in the salivary glands of I. ricinus female ticks, a representational difference analysis subtractive library was set up using mRNAs extracted from salivary glands of unfed and 5-day-fed female I. ricinus ticks (Leboulle et al, 2002). One clone, formerly named SEQ. ID. NO. 7 (GenBank_{—} accession no. AJ269641), was selected for further characterization of its recombinant protein, because of its similarity to the second kunitz-domain of the human tissue factor pathway inhibitor. Indeed, the amino sequence comprises the typical consensus kunitz motif F-x(3)-G-C-x(6)-[FY]-x(5)-C (FIG. 3A). The signal peptide sequence is in bold and underlined. The kuntiz motif sequence is shaded. Calculated MW and pI were 7,659 Da and 8.89, respectively. Moreover, SignalP and TargetP programs predicted a signal peptide cleavage site at position 23 and the absence of hydrophobic transmembrane region, suggesting that the protein was secreted. In order to find homologs, PDB was searched using the Blast algorithm. SEQ. ID. NO. 7 displayed 30% identity and 39% similarity with the kunitz-type chymotrypsin inhibitor from Bungarus fasciatus. Both shared some conserved residues (e.g., proline and glycine, and 6 cysteine residues predicted to form three disulphide bridges; FIG. 3B). FIG. 3 represents amino acid sequence comparison of SEQ. ID. NO. 7 (Ir-CPI with its peptide signal) with the kunitz-type chymotrypsin inhibitor from Bungarus fasciatus (BF9). Some shared conserved residues are shaded (P, praline residue; G, glycine residue). Three disulfide bridges are represented. Finally, no consensus sites for N- and O-glycosylation were predicted in the sequence.

In order to produce a recombinant form of SEQ. ID. NO. 7, its coding sequence, without its expected cleavage site and its peptide signal was cloned in the expression vector pGEX-6P-1 in-frame with the coding sequence of glutathione S-transferase and expressed in bacteria. Affinity purification followed by cleavage with PreScission protease and further fast protein liquid chromatography yielded pure protein.

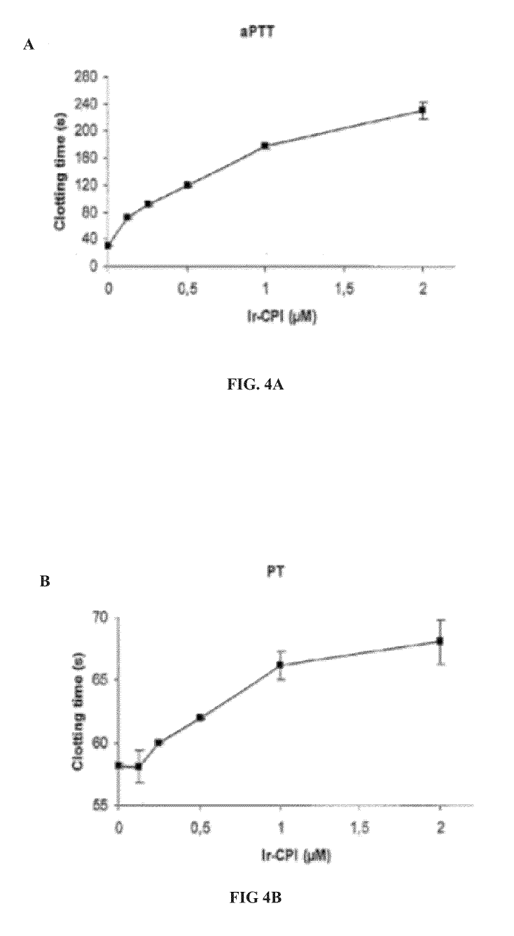

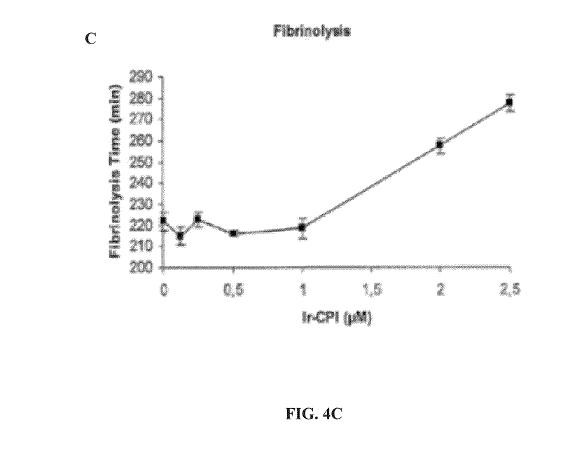

Ir-CPI prolongs activated partial thromboplastin (aPTT), prothrombin (Pt) and fibrinolysis times (FIG. 4)

The activity of recombinant Ir-CPI for “Ixodes ricinus Contact Phase Inhibitor” was analyzed on the three classical hemostasis pathways. No effect was observed on primary hemostasis for the two activators tested (collagen/epinephrine or collagen/ADP). For the two other pathways, the anticoagulant activity of Ir-CPI was determined by using four tests measuring plasma clotting times. Analysis of all the results showed that recombinant Ir-CPI prolongs aPTT (7.7 times at 2 μM) and PT (1.2 times at 2 μM) in a dose-dependent manner. The thrombin and stypven times were unchanged. The activity of Ir-CPI was also investigated on fibrinolysis. The results showed that the fibrinolysis time was slightly increased by 1.2 times in the presence of Ir-CPI at 2 μM. FIG. 4 represents the effects of Ir-CPI on aPTT, PT and Fibrinolysis times. Inhibitory activities of Ir-CPI was estimated on the intrinsic and extrinsic coagulation pathways, and on the fibrinolysis.

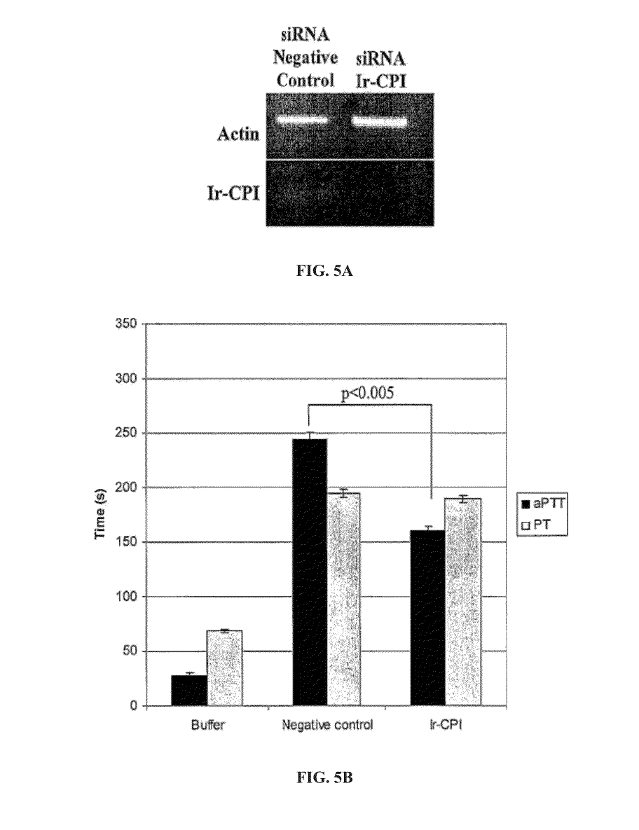

### Natural Ir-CPI has an Anticoagulant Activity (FIG. 5)

The “RNA interference” method makes it possible to study the properties and role of a protein in its natural context. The inventors therefore synthesized siRNAs specific for Ir-CPI mRNA. The specificity of this siRNA was measured by RT-PCR on salivary gland mRNA extracts. The results showed that Ir-CPI mRNA was only silenced in SGE treated with Ir-CPI siRNA (FIG. 5A). FIG. 5A represents evaluation of the Ir-CPI siRNA specificity by RT-PCR. Salivary glands from 5-days fed female ticks were incubated with siRNA negative control duplexes, actin siRNA or Ir-CPI siRNA for 6 h at 37° C. RT-PCR assays were then realized by using action or Ir-CPI gene specific primers. The inventors then measured the effect on aPTT and PT of these siRNA-treated salivary gland extracts (FIG. 5B). FIG. 5B represents the effect of Ir-CPI siRNA-treated salivary gland extracts on aPTT and PT. Salivary glands were incubated either with negative control siRNA (negative control) or with Ir-CPI siRNA (Ir-CPI). The effect of these siRNA-treated salivary gland extracts on coagulation time (aPTT, PT) was then examined in aPTT and PT assays. Human plasma incubated with buffer served as a control (Buffer). Statistical significance was calculated by using one-way Anova and Student-Newman-Keuls test.

Salivary gland extracts incubated with siRNA negative control had a mean aPTT of 217.2 s and PT of 125.8 s. When the same quantity of SGE was treated with Ir-CPI-specific siRNA, there was a major fall in aPTT and a minor fall in PT to values of 132.7 s and 121 s respectively.

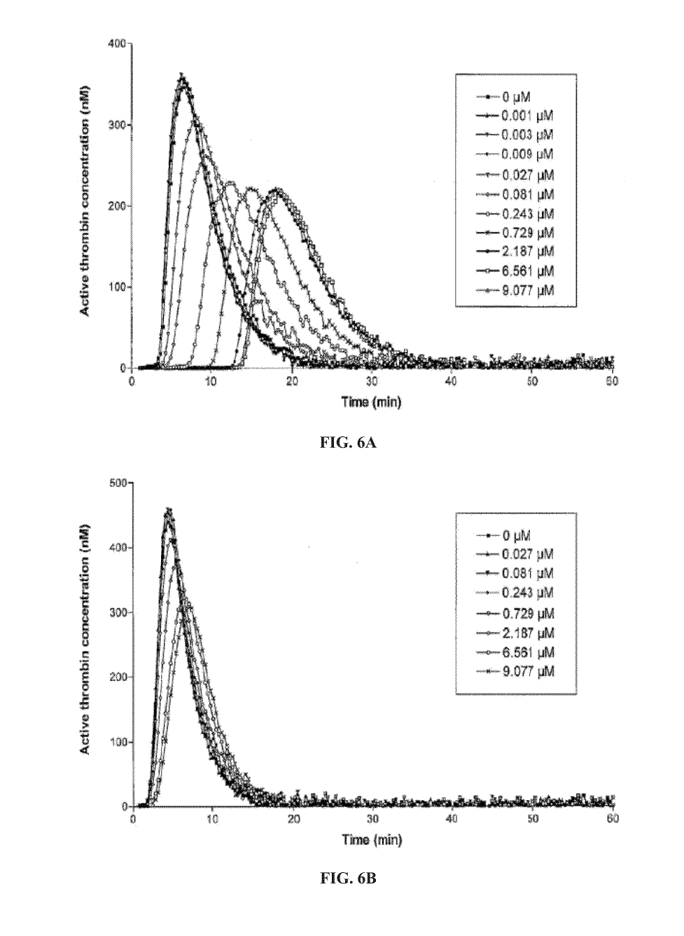

### Ir-CPI Inhibits Thrombin Generation (FIG. 6)

The effects of Ir-CPI were first investigated on thrombin activity during coagulation process induced by the intrinsic pathway by using a mixture of ellagic acid and phospholipids as trigger. Ir-CPI caused a dose-dependent prolongation of the lag time and a dose-dependent decrease of the maximal concentration of active thrombin (Cmax) compared to the control curve (i.e. without inhibitor) (FIG. 6A). At 9.077 μM, the lag time was prolonged 3.6 fold compared to the control curve. Regarding the Cmax, the effect was maximal at 2.187 μM and did not increase at higher concentrations (6.561 μM; 9.077 μM). At this concentration, the Cmax was reduced by 37% and the lag time was prolonged 2.7 fold.

When coagulation cascade was triggered by the extrinsic pathway (5 pM of tissue factor (TF) and 4 μM of phospholipids (PL)), a slight dose-dependent decrease of the Cmax and a dose-dependent prolongation of the lag time were found (FIG. 6B). At 9.077 μM, the Cmax was reduced by 30% and the lag time was prolonged 1.6 fold. Similar results were obtained when using a lower concentration of TF (1 pM) and 4 μM PL. FIG. 6 represents the effect of Ir-CPI on thrombin activity profile during coagulation process induced by either ellagic acid and PL (A) or 5 pM TF and PL (B).

Taken together, these results confirm that Ir-CPI is a potent inhibitor of the thrombin generation induced by the intrinsic pathway, and to a lower extent by the extrinsic pathway.

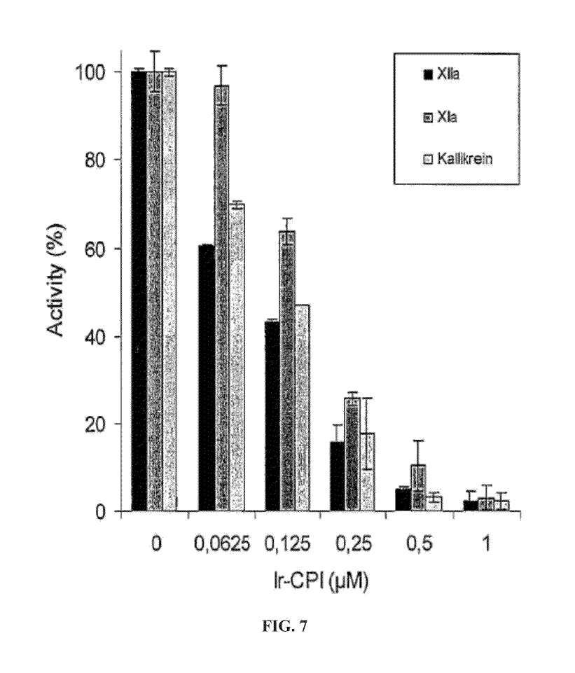

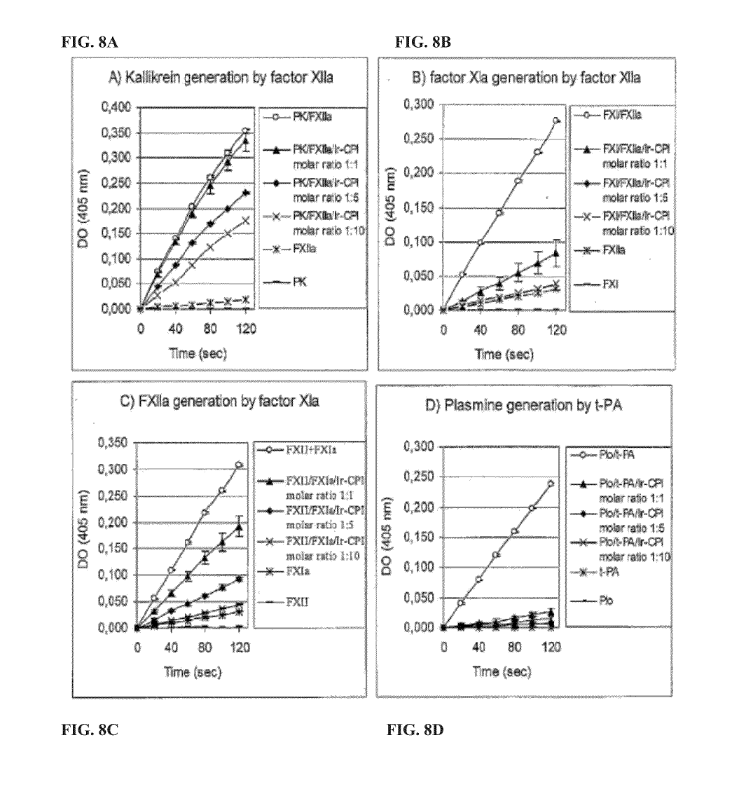

### Ir-CPI Inhibits the Activation of Contact System Factors (FIGS. 7 and 8)

In order to determine the target of Ir-CPI, the effect of Ir-CPI on 7 procoagulant serine proteases (kallikrein, Factor XIIa, XIa, IXa, IXa, Xa, IIa and VIIa) and 2 fibrinolytic serine proteases (t-PA and plasmin) was measured with amidolytic tests using the specific substrate of each of these serine proteases. The results obtained did not show any effect of Ir-CPI protein on the amidolytic activity of these factors. FIG. 7 represents the the inhibitory effect of Ir-CPI on generation of factor XIIa, factor XIa and kallikrein in human plasma. Diluted human plasma was incubated with various concentrations of Ir-CPI (0.0625, 0.125, 0.25, 0.5 and 1 μM), and the mixture was activated with aPTT reagent to initiate the contact system. The amidolytic activities of generated factor XIIa, Factor XIa and kallikrein were determiner by addition of chromogenic substrate, and increases in absorbance at 405 nm were recorded. Results are presented as the mean±SD of triplicate determinations.

The capacity of Ir-CPI protein to inhibit the activation of human plasma contact factors was then analyzed. In this experiment, human plasma was preincubated with Ir-CPI and then treated with a contact phase activator. The activation of contact factors (factor XIIa, XIa and kallikrein) was then evaluated by using the specific substrate of each factor. The results showed that Ir-CPI inhibits the generation of these three factors in a dose-dependent manner.

The effect of Ir-CPI was then examined in different reconstituted systems by using purified factors and their associated chromogenic substrates. In each of these experiments, the inventors analyzed the activation of a non-activated factor by an activated factor, in the presence or absence of Ir-CPI. The results showed that Ir-CPI inhibits the activation of prekallikrein into kallikrein by factor XIIa, the activation of factor XI into factor XIa by factor XIIa and the activation of factor XII into factor XIIa by factor XIa. On the contrary, Ir-CPI did not inhibit the activation of factor XII into factor XIIa by kallikrein or the activation of factor X into factor Xa by tissue factor complex/factor VIIa though it did inhibit the activation of plasminogen into plasmin by t-PA. FIG. 8 represents the inhibitory effect of Ir-CPI on reconstituted systems. The effect of Ir-CPI was examined in different reconstituted systems by using purified factors and their associated chromogenic substrates. The activation of a non-activated factor by an activated factor, in the presence or absence of Ir-CPI was analyzed in each experiment.

Taken overall, the results of these experiments show that Ir-CPI has a major effect on the activated factors participating in the contact phase of coagulation.

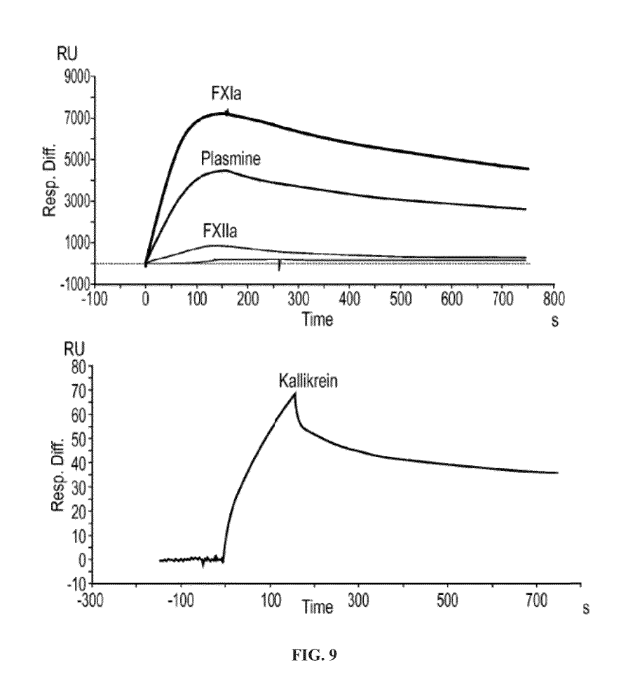

Ir-CPI Binds to Factor XIa, fXIIa, Kallikrein and Plasmin (FIG. 9)

The ability of Ir-CPI to bind a (co)factor of coagulation or fibrinolysis was evaluated by surface plasmon resonance. The results demonstrated a specific interaction between Ir-CPI and four factors: fXIIa, fXIa, plasmin and kallikrein. No interaction was observed for the other (co)factors tested (prekallikrein, HMWK, fXII, fXI, fIX, fIXa, fX, fXa, thrombin, fVIIa, t-PA and plasminogen). Moreover, the kinetics of interaction between Ir-CPI and the four target factors (XIIa, XIa, plasmin and kallikrein) were measured after a new immobilization of Ir-CPI. In experiments to determine the binding kinetics, the quantity of immobilized Ir-CPI was deliberately kept at a low level (approximately 200 RU) in order to avoid problems where the reaction rate is limited by mass-transport The initial binding rate was shown to be independent of variations in flow by linear regression measurements at the start of kinetics with injections of analytes at increasing flows from 30 to 70 μl/min, confirming that there was no limitation of the reaction. Interaction kinetics were determined for each analyte, at 6 different concentrations (from 5 nM to 300 nM). The kinetic data obtained were individually processed with BIA evaluation software in order to determine the kinetics constants. The results obtained in this way showed that the affinity constant (Kd) of Ir-CPI was similar for fXIIa, fXIa, and plasmin (about nM: from 1.81 to 5.89 nM) whereas it was lower for kallikrein (0.2 μM). FIG. 9 represents sensorgrams for interactions between coagulation factors and immobilized Ir-CPI measured by surface plasmon resonance. Ir-CPI was immobilized onto the surface of a sensor chip CM5 at level of 1500 resonance units (RUs). Contact factors (100 nM final concentration) were injected at a flow rate of 70 μl/min in HBS buffer, and association was monitored. After return to buffer flow, dissociation was monitored during 84 s. The sensor chip surface was regenerated by a pulse injection of 25 mM NaOH after each experiment.

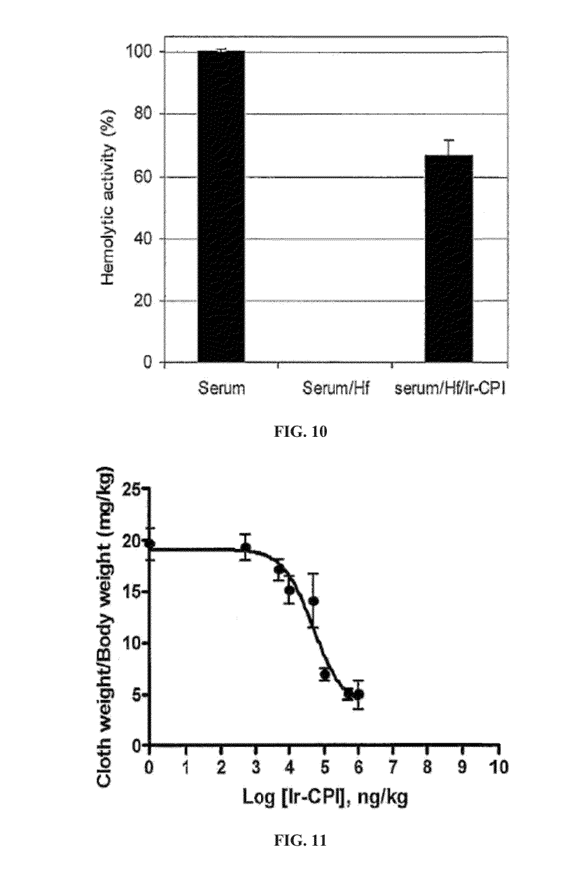

Ir-CPI does not Inhibit the Classical and Alternative Complement Pathways; but Inhibits the Activation of Complement Factor C1 (FIG. 10)

The inventors also measured the direct effect of recombinant Ir-CPI on the alternative and classical complement pathways using red blood cell hemolysis tests. The results showed that there was no significant effect of Ir-CPI on these 2 pathways indicating that Ir-CPI does not directly interact with any of the factors of these 2 pathways. However, the inventors also examined the capacity of Ir-CPI to inhibit the activation of the classical complement pathway by fragment f of factor XII (factor Hf). In this experiment, Hf was preincubated with Ir-CPI before adding human serum. Under normal conditions, the incubation of Hf with normal serum leads to the sequential depletion of serum C1, C4, C2, and C3 following the activation of the classical complement pathway. In the presence of Ir-CPI, the inventors observed that Ir-CPI inhibits the initiation of the classical complement pathway via factor Hf. FIG. 10 represents inhibitory effect of Ir-CPI on the activation of the classical complement pathway by fragment f of factor XII (factor Hf). Hf was preincubated in the presence (Serum/Hf/Ir-CPI) or absence (Serum/Hf) of Ir-CPI before adding human serum. The mixture was then incubated with sensitized sheep erythrocytes for 60 min at 37° C. The reaction was stopped by addition of 150 μl of NaCl 0.9%, the mixture was centrifuged, and free hemoglobin was measured in the supernatant at 415 nm Results are presented as the mean±SD of triplicate determinations.

### Effect of Ir-CPI on Stasis-Induced Venous Thrombosis in Rats.

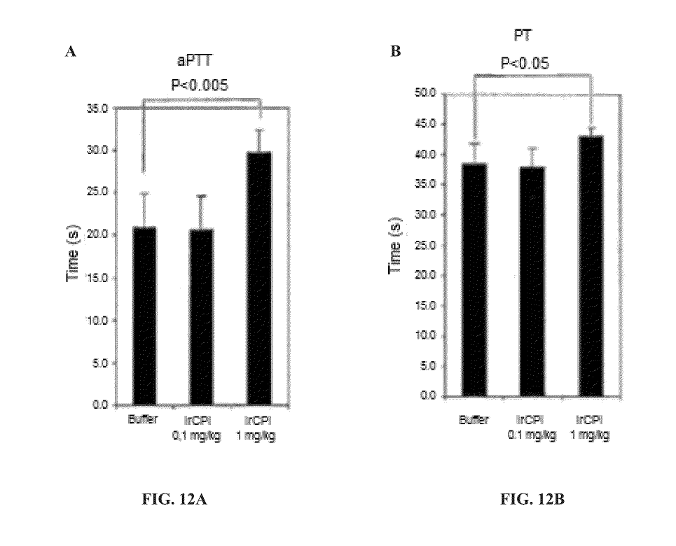

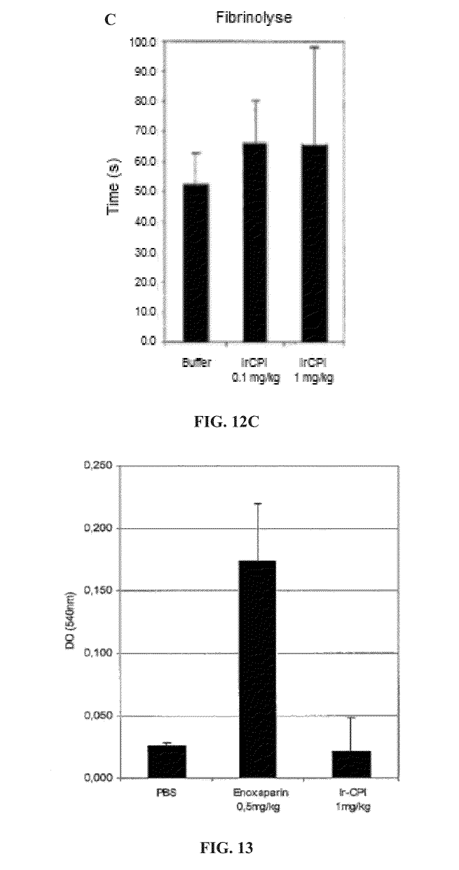

To determine whether Ir-CPI has an antithrombotic action in vivo, we used a venous thrombosis model in rats that combines stasis by vessel ligation and activation of thrombosis by severe endothelial damage and vessel occlusion with ferric chloride (see Materials and Methods). The control group showed 100% thrombus formation, with a mean thrombus weight of 19.6±1.6 mg/kg (n=6). In contrast, intravenous administration of Ir-CPI induced a progressive decrease in thrombus formation, with EC50 at about 50 μg/kg and with a maximum effect starting at 100 μg/kg (FIG. 11). FIG. 11 represents the effect of Ir-CPI on stasis-induced venous thrombosis in rats. Ir-CPI at the indicated doses was administered i.v. 5 min before induction of thrombosis by 10% FeCl3 and complete stasis, as described in Materials and methods. The control group received PBS instead of Ir-CPI. Each point represents mean±SD of five to six animals. In addition, the inventors also evaluated the half-life of Ir-CPI in vivo. A semi-quantitative estimate of Ir-CPI pharmacokinetics was obtained using ¹²⁵I-Ir-CPI. The result shows that plasma ¹²⁵I-Ir-CPI concentrations reached a peak 3 h after intravenous administration and were about 40.8%±9.9% of the maximum value 20 h after administration of the recombinant protein. The effects of Ir-CPI on ex vivo clotting assays were then tested. FIG. 12 represents ex vivo anticoagulant and fibrinolysis activity of Ir-CPI. Ir-CPI at the indicated concentrations was given intravenously to rats; after 5 min, blood was collected, and platelet-poor plasma was obtained. Coagulation tests aPTT, PT, fibrinolysis time were determined as described in Materials and methods. Each point represents mean±SD of five animals.

FIG. 12 shows that aPTT values were similar in comparison with controls for Ir-CPI EC50 and 100 μg/kg doses whereas aPTT values were statistically higher in comparison with controls for Ir-CPI doses higher than 1 mg/kg, showing a ˜1.4-fold increase in that case. In contrast, PT was only slightly affected by 1 mg/kg Ir-CPI. Moreover, this high dose of Ir-CPI had no effect on the fibrinolysis time. FIG. 13 represents the determination of the bleeding effect of Ir-CPI. Ir-CPI at the indicated dose was administered intravenously; after 5 min of administration, the rat tail was cut 3 mm from the tip. The tail was carefully immersed in 10 ml of distilled water at room temperature, and blood loss (hemoglobin content) was estimated at 540 nm after 60 min. The absorbance detected for a group that received PBS or Enoxaparin instead of Ir-CPI was taken as controls. Results represent the mean±SD of five animals. Finally, the bleeding effect of Ir-CPI was evaluated using a tail-transection model (FIG. 13); no statistically significant blood loss was observed 5 min after administration of 1 mg/kg Ir-CPI.

The coagulation cascade occurring in mammalian plasma involves a large number of plasma proteins that participate in a stepwise manner and eventually lead to the generation of thrombin. This enzyme then converts fibrinogen to an insoluble fibrin clot. Blood coagulation starts immediately after damage to the vascular endothelium and uncovering of the subendothelial structures. Contact phase proteins include the zymogens, factor XII, prekallikrein, factor XI and the cofactor, high molecular weight kininogen (HMWK). Factor XII autoactivates when bound to polyanionic surfaces, with conversion of factor XII to factor XIIa. Surface-bound activated factor XII then converts prekallikrein into kallikrein by cleavage of a single peptide bond. However, once small amounts of kallikrein are formed, there is rapid conversion of surface-bound factor XII to factor XIIa, resulting in strong positive feedback on the system. During activation of proenzymes, factor XII may also be activated during proteolysis by kallikrein leading to the production of a series of active enzymes formed by successive cleavages. Kallikrein first cleaves surface bound single-chain factor XII into a two-chain active α-factor XIIa. The newly formed α-factor XIIa has the same molecular weight as zymogen but is composed of a heavy chain of 50 kDa and a light chain of 28 kDa. The intrinsic coagulation pathway is initiated by cleavage of factor XI into activated factor XI (factor XIa) by α-factor XIIa. The heavy chain may be further cleaved into a series of lower molecular-weight forms of activated XII, known as Hageman factor fragment (HFf), all of which retain activity in terms of conversion of prekallikrein to kallikrein but lose the ability to activate factor XI. Similarly, HFf will not activate zymogen factor XII and therefore does not participate in autoactivation.

It later became clear that activation of the contact-phase system plays an essential role in fibrinolysis as it results in the activation of plasmin and pro-urokinase.

Serine protease, which is generated after initiation of the intrinsic pathway, also influences complement. Thus, plasmin, factor HFf, and kallikrein are responsible for activation of the C1r and C1 s subunits of the first complement component, which are precursors of serine proteinases in the classic activation pathway and factor B, which is a proform of the serine proteinase of the alternative complement activation pathway.

Moreover, kallikrein is an activator of prorenin and is responsible for kinin formation. The contact phase has therefore been shown to initiate activation not only of the coagulation system but also of all the other proteolytic systems in blood plasma: kallikrein-kinin, complement, fibrinolytic, and renin-angiotensin systems. HFf can also activate factor VII, the proenzyme initiating the extrinsic coagulation pathway, dependent on tissue factor (TF).

Many blood-sucking ectoparasites synthesize substances to thwart the defense mechanisms of the hosts on which they feed. In order to effectively acquire and digest their blood meal, ticks must adapt to their host's defense systems and produce a certain number of salivary substances capable of modulating the host immune responses and maintaining blood in a sufficiently fluid state to acquire this meal.

The hemostatic system is composed of a network of factors, and the activation of each pathway may be induced in many different ways. Ticks however are confronted with the problem of redundancy as it is not sufficient to specifically inhibit a single factor as another pathway may take its place and activate blood clotting. However, the long parallel tick/host evolution has allowed ticks to confront such a system by producing several compounds with an anti-hemostatic activity.

When ticks take a blood meal, the action of the chelicerae and insertion of the hypostome into the host skin causes damage to the epidermis and dermis with rupture of local blood vessels thereby activating the contact phase pathway. Few inhibitors that act contact phase pathway have so far been discovered. Haemaphysalin is an inhibitor of the kallikrein-kinin system with two kunitz domains discovered in Haemaphysalis longicornis (Kato et al., (Thrombosis haemostasis, vol 93 p 359-367) 2005a). It appears that this molecule binds via its C terminal domain to the cell binding domains of high molecular weight kininogen and also that of fXIIa, which prevents the activation stages of the compounds of the contact system (Kato et al., (Journal of Biochemistry vol 38 (3) p 225-235) 2005b).

Ir-CPI is a low molecular weight protein that plays a major role in the tick blood meal by interfering with the activated factors involved in the contact phase of the coagulant balance. Such an inhibitor is not unexpected as the tick uses its chelicerae, pedipalps and hypostome during feeding. These cause extensive damage to the tissues surrounding the bite site by locally breaking the vessels and establishing a nutrition cavity rich in cells and in host blood factors. This phenomenon leads to the activation of contact phase factors.

Autoactivation of factor XII into factor XIIa usually occurs during the contact phase. This may therefore trigger both the intrinsic coagulation pathway by activating factor XI and also an inflammatory process by activating prekallikrein into kallikrein. Then, once activated, kallikrein releases bradykinin from high molecular weight kininogen. Bradykinin is an endogenous polypeptide comprising nine amino-acids. Bradykinin is one of the most potent vasodilators known which increases capillary permeability and promotes the development of edema. In addition to kallikrein, other tissue or plasma proteases are capable of cleaving bradykinin and others kinins. Plasmin, which is responsible for lysis of the fibrin clot, releases not only bradykinin but also its derivatives. Moreover, factor XIIa, XIa, and kallikrein are also capable of converting plasminogen into plasmin By directly acting on factors XIIa and XIa, Ir-CPI blocks the intrinsic coagulation pathway; but also prevents the formation of kallikrein which plays an active role in the amplification process of these two factors. The inhibition of kallikrein production makes it possible to prevent the initiation of an inflammatory process by bradykinin release. Moreover, bradykinin production is also blocked by direct inhibition of plasmin and indirect inhibition of factor XIIa, XIa and kallikrein which are no longer capable of activating plasminogen into plasmin.

Factor XIIa also has an important role in the activation of the complement system. Factor XIIa can activate C1r and to a lesser extent C1s, the first element of the complement cascade. The hemolysis assay of the classical complement pathway using sheep red blood cells demonstrated the capacity of Ir-CPI to inhibit the initiation of this pathway by factor XIIa via factor Hf.

Moreover, deficiencies in these factors (XII, XI and prekallikrein) do not give rise to clinical situations that may be explained by impaired clotting or fibrinolysis. The coagulation balance of factor XII-knockout mice and all-deficient patients is not disturbed in any way and is similar to that observed in wild mice and healthy patients. On the other hand, fXII-KO mice are protected from thrombus formation, an essential element in venous thrombosis, cerebral ischemia and arterial thrombosis. The preclinical evaluation of Ir-CPI in 2 models of venous thrombosis suggests that Ir-CPI may therefore mirror the situation in KO mice by preventing clot formation without interfering with the clotting equilibrium (aPTT, PT and fibrinolysis were unchanged at the effective dose) or with the bleeding time. Ir-CPI therefore provide an excellent therapeutic tool by protecting patients at risk from diseases such as pulmonary embolism, cerebral ischemia or deep vein thrombosis.

### Example 10

### Effect of Ir-CPI on FXI and FXII Coagulation Activities

### Method

The effect of Ir-CPI on the activity of the coagulation factors XI and XII in human plasma was investigated using an aPTT-based assay.

Nine volumes of a human plasma were mixed with one volume of Ir-CPI and incubated during 30 minutes at 37° C. Ir-CPI was added to plasma as a 10-fold solution to obtain final Ir-CPI concentrations ranging from 0.125 to 20 μg/mL. Ir-CPI treatment was compared to control (i.e. absence of Ir-CPI) consisting of nine volumes of the human plasma to which one volume of physiological saline (0.15 M NaCl) was added. After incubation, samples were diluted 1:10 with imidazole buffer. Then, 100 μL of each diluted sample was mixed with 100 μL of Factor XI deficient human plasma or with 100 μL Factor XII deficient human plasma, followed by an addition of 100 μL of the aPTT reagent Cephen. The contact phase was activated by Cephen during an incubation of 10 min at 37° C. Clotting was initiated by the addition of 100 μL of 0.025 M CaCl₂ and clotting times were recorded.

Calibration curves were made using successive two-step dilutions of human plasma (ranging from 1:10 to 1:160) using imidazole buffer. Clotting times were plotted in function of the FXI/FXII activity of the different dilutions of plasma using a log-log plot. The 10-fold dilution of the plasma is considered having 100% activity. There is an inverse linear relationship between the FXI or FXII activity and the corresponding clotting time when plotted on a log-log-graph. The equations of the calibration curves were used to calculate the FXI and FXII activities in human plasma treated with Ir-CPI. The residual activity after Ir-CPI treatment was calculated as a percentage compared to control (untreated normal human plasma). Results are expressed as the percentage of inhibition of FXI and FXII activities.

Data were analyzed according to the fitting to a hyperbolic equation assuming maximal inhibition of 100%:

$$E = \frac{{E}_{max} * [ C ]}{{EC}_{max / 2} + [ C ]} = \frac{100 * [ C ]}{EC 50 + [ C ]}$$

E=Effect in % of inhibition
Eₘₐₓ=Maximum % of inhibition, fixed at 100%
EC_{max/2}=Concentration producing half of maximal effect
EC50=Concentration producing an inhibition of 50%
[C]=Concentration of inhibitor (μg/mL)

### Results

Three independent assays were performed using human FXI or FXII deficient plasmas complemented with normal human plasma treated with variable concentrations (0 to 20 μg/mL) of Ir-CPI.

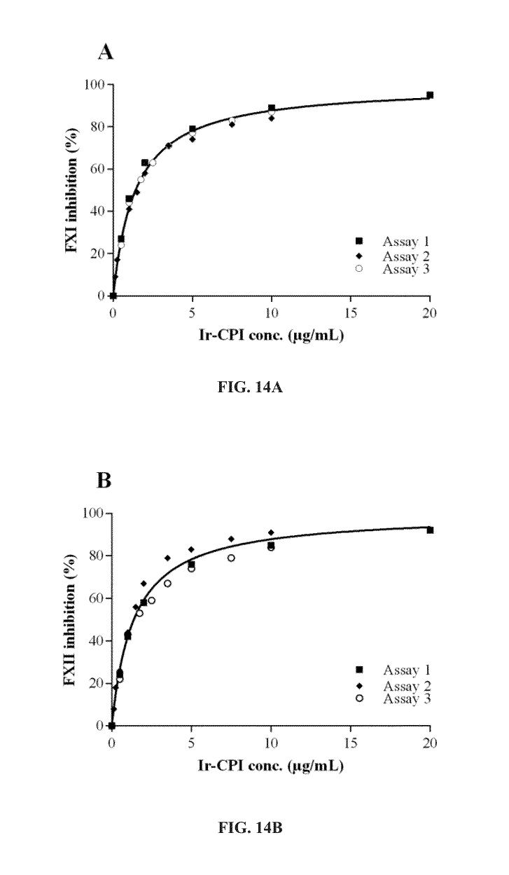

As shown in FIG. 14, Ir-CPI inhibits similarly FXI and FXII coagulation activities in this human plasma assay. EC50 was 1.41 μg/mL and 1.38 μg/mL regarding inhibition of FXI and FXII coagulation activities, respectively with a maximal effect of almost 100% on both coagulation factors (Table 3).

TABLE 3
Inhibition of FXI and FXII coagulation activities. Parameters calculated
based on a fitting to a hyberbolic equation.

| | FXI | FXII |
|---|---|---|
| Emax (%) | 100 | 100 |
| EC50 (μg/mL) | 1.41 | 1.38 |
| CI95% (μg/mL) | 1.34-1.47 | 1.27-1.49 |

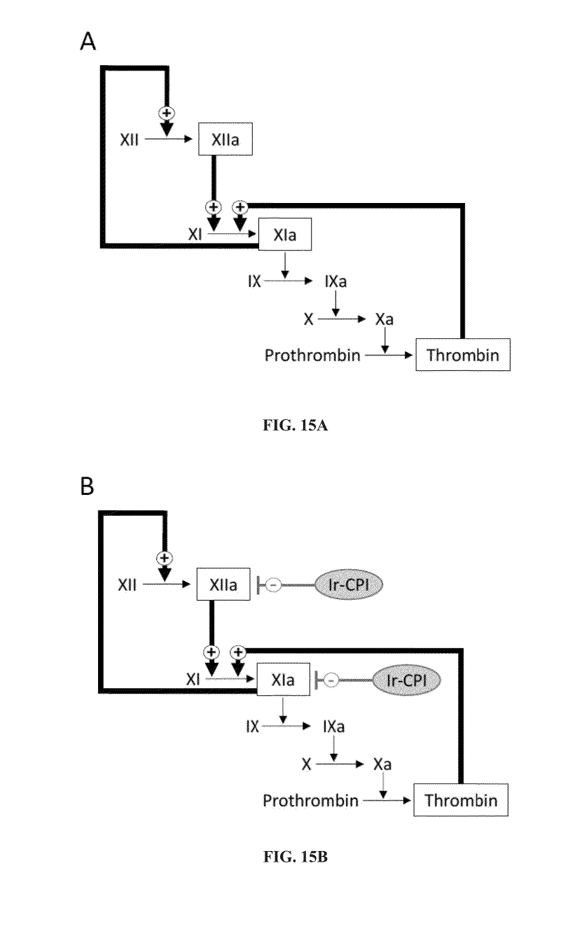

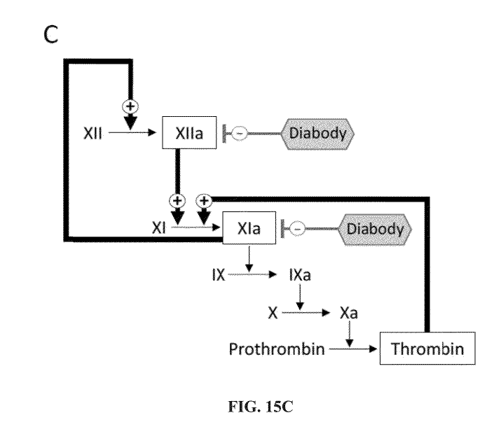

As schematically illustrated in FIG. 15A, factor XIIa activates factor XI to XIa and factor XIa activates factor XII to XIIa. These mutual activations contribute to the amplification of the coagulation signal of the intrinsic coagulation phase and ultimately to the formation of thrombin. Factor XI can also be activated by thrombin, thus independently of factor XIIa. Activation of factor XI by thrombin is also thought to play a major role as a feedback loop of amplification of the intrinsic pathway of coagulation.

Three independent experimental approaches, i.e. reconstituted systems (see Example 9, FIG. 8), surface plasmon resonance (see Example 9, FIG. 9), and aPTT-based coagulation assay (see Example 10, FIG. 14), confirm that Ir-CPI is a dual inhibitor of factors XIa and XIIa. Ir-CPI is a potent and unique contact phase inhibitor because it inhibits two key factors (FXI and FXII) involved in the activation but also the amplification of the intrinsic coagulation pathway (FIG. 15B). Thanks to this dual activity, Ir-CPI may be expected to have an improved anti-thrombotic/anticoagulant efficacy compared to drugs targeting factor XI or factor XII alone. Similar therapeutical advantages may be expected using a heterodimer, bispecific diabody targeting both factor XI and factor XII (FIG. 15C).

### Example 11

### Characterisation and Determination of the Anticoagulant Potential of Diabodies Directed Against Factor XI and Factor XII Activities

The binding interaction between diabodies and factors XI, XIa, XII and XIIa are monitored by surface plasmon resonance according to the method previously described in Example 9.

The functional effects of diabodies on coagulation activities associated to factor XI or/and factor XII are monitored using human plasma deficient in factor XI or in factor XII, respectively and complemented with diluted human plasma according to the method previously described in Example 10.

1. An inhibitor of a plasma contact factor, wherein said inhibitor is an isolated polypeptide having less than 100% and at least 75% sequence identity to the amino acid SEQ ID NO: 36 or a diabody.

2. The inhibitor according to claim 1, wherein said inhibition is selected from the group comprising inhibition of the activation of factor XI into factor XIa by factor XIIa, inhibition of the activation of factor XII into factor XIIa by factor XIa, or a combination thereof.

3. The inhibitor according to claim 1, wherein said isolated polypeptide comprises at least 80% sequence identity to the amino acid SEQ ID NO: 36.

4. The inhibitor according to claim 1, wherein said isolated polypeptide comprises at least 90% sequence identity to the amino acid SEQ ID NO: 36.

5. The inhibitor according to claim 1, wherein said isolated polypeptide comprises at least 95% sequence identity to the amino acid SEQ ID NO: 36.

6. The inhibitor according to claim 1, wherein said isolated polypeptide comprises at least one substitution group.

7. The inhibitor according to claim 1, wherein said isolated polypeptide is selected from the group consisting of a polypeptide having up to 5 amino acids substitutions relative to the amino acid sequence of SEQ ID NO: 36, a polypeptide having up to 5 amino acids deletions relative to the amino acid sequence of SEQ ID NO: 36, and a polypeptide having up to 5 amino acids additions relative to the amino acid sequence of SEQ ID NO: 36.

8. The inhibitor according to claim 1, wherein said isolated polypeptide is a polypeptide having at least 95% sequence identity to the amino acid sequence of SEQ ID NO:36, wherein said polypeptide has a kunitz-type-protease-inhibitor (KPI) domain, wherein the KPI domain of the polypeptide comprises Phe at position corresponding to position 40 of SEQ ID NO:36, Gly at position corresponding to position 44 of SEQ ID NO:36; Cys at position corresponding to position 45 of SEQ ID NO:36, Phe at position corresponding to position 52 of SEQ ID NO:36, and Cys at position corresponding to position 58 of SEQ ID NO:36.

9. The inhibitor according to claim 1, wherein said isolated polypeptide is fused to a heterologous polypeptide.

10. The inhibitor according to claim 9, wherein said heterologous polypeptide comprises multiple histidine residues.

11. The inhibitor according to claim 1, wherein said isolated polypeptide is a polypeptide having at least 95% sequence identity to the amino acid sequence of SEQ ID NO:36 fused to a heterologous polypeptide, wherein said polypeptide has a kunitz-type-protease-inhibitor (KPI) domain, wherein the KPI domain of the polypeptide comprises Phe at position corresponding to position 40 of SEQ ID NO:36, Gly at position corresponding to position 44 of SEQ ID NO:36; Cys at position corresponding to position 45 of SEQ ID NO:36, Phe at position corresponding to position 52 of SEQ ID NO:36, and Cys at position corresponding to position 58 of SEQ ID NO:36.

12. The inhibitor according to claim 1, wherein said diabody recognizes two different polypeptides from the group comprising factor XI, factor XII, factor XIa and factor XIIa.

13. A method for preventing and/or treating a plasma contact factor-related disease comprising administration of an inhibitor of a plasma contact factor in a subject in need thereof, wherein said inhibitor is an isolated polypeptide having less than 100% and at least 75% sequence identity to the amino acid SEQ ID NO: 36 or a diabody.

14. The method for preventing and/or treating a plasma contact factor-related disease according to claim 13, wherein said inhibition is selected from the group comprising inhibition of the activation of factor XI into factor XIa by factor XIIa, inhibition of the activation of factor XII into factor XIIa by factor XIa, or a combination thereof.

15. The method for preventing and/or treating a plasma contact factor-related disease according to claim 13, wherein said plasma contact factor-related disease is selected from the group comprising deep vein thrombosis, portal vein thrombosis, jugular vein thrombosis, renal vein thrombosis, pulmonary embolism, unstable angina, acute coronary syndrome, myocardial infraction, cerebral ischemia and stroke.

16. The method for preventing and/or treating a plasma contact factor-related disease according to claim 13, wherein said plasma contact factor-related disease is the thrombus formation during and/or after the contact of blood with artificial surfaces.

17. The method for preventing and/or treating a plasma contact factor-related disease according to claim 13, wherein said plasma contact factor-related disease is the thrombus formation during and/or after a medical procedure such as comprising extracorporeal membrane oxygenation for blood oxygenation, extracorporeal circulation during cardiopulmonary bypass, dialysis and extracorporeal filtration of blood, percutaneous angioplasty, use intraluminal catheters and stents, intra-aortic balloon pump.
